(19) United States
(12) Patent Application Publication
(10) Pub. No.: US 2025/0304985 A1
(43) Pub. Date: Oct. 2, 2025

(54) Genetically Engineered Plants for Increased Production of Vindoline

(71) Applicant: Northeastern University, Boston, MA (US)

(72) Inventors: Carolyn LEE-PARSONS, Boston, MA (US); Lauren F. COLE, Cambridge, MA (US)

(21) Appl. No.: 18/848,162

(22) PCT Filed: Apr. 10, 2023
(86) PCT No.: PCT/US2023/018093

§ 371 Date: Sep. 18, 2024

Related U.S. Application Data
(60) Provisional application No. 63/329,348, filed on Apr. 8, 2022.

(51) Int. Cl. C12N 15/82; C07K 14/415; C12P 17/12

(52) U.S. Cl. CPC C12N 15/8243; C07K 14/415; C12P 17/12

(57) ABSTRACT

Two novel transcription factors, CrDELLA1 and CrDELLA2, are described in Catharanthus roseus plants. The DELLA transcription factors have a regulatory role in the synthesis of vinblastine and vincristine, two important anti-cancer compounds that have proved difficult to obtain in sufficient quantities. The present technology provides genetically modified C. roseus plants having enhanced DELLA activity, which leads to activation of multiple enzymes in the biosynthetic pathway leading to vinblastine and vincristine. The genetic modifications can be used together with activation of plant defense mechanisms and responses to light in order to boost vinblastine and vincristine synthesis.

### CROSS REFERENCE TO RELATED APPLICATIONS

This application claims the priority of U.S. Provisional Application No. 63/329,348 filed 8 Apr. 2022 and entitled “Genetically Engineered Plants for Increased Production of Vindoline”, the whole of which is hereby incorporated by reference.

### STATEMENT REGARDING FEDERALLY SPONSORED RESEARCH OR DEVELOPMENT

This Invention was made with government support under Grant No. 1516371 awarded by the National Science Foundation. The government has certain rights in the invention.

### BACKGROUND

Catharanthus roseus is a rich source of terpenoid indole alkaloids (TIAs), including the valuable chemotherapy medicines, vinblastine (VBL) and vincristine (VCR); these medicines are exclusively produced in trace amounts in the leaves of C. roseus. The biosynthetic pathway leading to VBL and VCR is complex, requiring over 30 enzymes, transport within cellular compartments and among multiple cell-types, and competing flux towards variable end-products (recently reviewed in [1]). VBL and VCR are derived from the universal TIA precursor, strictosidine, which is formed by the coupling of the indole, tryptamine, and the terpenoid, secologanin. Strictosidine forms a reactive aglycon, which branches into many derivatives, including vindoline and catharanthine. Vindoline and catharanthine couple to form anhydrovinblastine, a precursor to VBL and VCR. Identification and overexpression of transcription factors that regulate multiple steps in TIA biosynthesis is a promising strategy for increasing flux towards VBL and VCR.

Previous research efforts have characterized signaling pathways responsible for inducing TIA biosynthesis with the defense-associated phytohormone, jasmonic acid (JA). When a plant is attacked by an herbivore, it synthesizes JA, which is perceived by CORONATINE INSENSITIVE (COI). In the presence of JA, COI binds to JASMONATE ZIM DOMAIN (JAZ) proteins and signals for their degradation. JAZ proteins bind and repress MYC2, an activator of defense-associated genes, including many TIA biosynthesis genes [2-6]. In C. roseus, CrMYC2 induces the expression of transcription factors that also activate TIA biosynthesis, including OCTADECANOID-RESPONSIVE CATHARANTHUS AP2-DOMAIN (ORCAs) and BHLH IRIDOID SYNTHESIS (BISs) [6-14]. The transient and combinatorial overexpression of these transcription factors increased strictosidine, root-specific downstream products like horhammericine, and vindoline pathway intermediates like 16-hydroxytabersonine. However, vindoline, catharanthine, and vinblastine levels did not increase with overexpression of these three transcription factors [6, 14], indicating that these downstream pathways are regulated by different transcription factors than the upstream and root-specific pathways. A co-expression analysis of TIA biosynthetic gene expression under varying environmental conditions showed that the vindoline pathway (the seven enzymes responsible for converting tabersonine to vindoline—T16H, 16OMT, T3O, T3R, NMT, D4H, DAT) clustered separately from the rest of the TIA pathway, highlighting its unique transcriptional regulation [14]. Identification of transcription factors that regulate the vindoline pathway could potentially overcome this bottleneck in the production of VBL and VCR.

Vindoline pathway gene expression is unique compared to the rest of the TIA pathway because it is highly tissue-specific, mostly expressed in immature leaves [15, 16], and it is strongly activated by light [17-20]. In the presence of red light, phytochrome (Phy) relocates from the cytoplasm to the nucleus, where it phosphorylates the transcription factors, PHYTOCHROME INTERACTING FACTORS (PIFs), leading to their degradation, and causing a cascade of transcriptional changes [21, 22]. Recently, Liu at al. identified CrPIF1 as a repressor and CrGATA1 as an activator of all light-inducible vindoline pathway genes (T16H2, T3O, T3R, D4H, and DAT) [20].

Vindoline pathway gene expression is also inducible by JA [15, 16, 31, 23-30], but this inducibility is highly dependent on tissue-specificity, light, and developmental state. For example, JA induced vindoline accumulation when applied to very young seedlings [32, 33] or multiple shoot cultures [29, 34], but not when applied to older seedlings or mature plants [35-39]. When caterpilars fed on mature C. roseus plants, inducing endogenous JA synthesis, strictosidine levels increased rapidly within a day, but vindoline and catharanthine levels only increased in emerging leaves a week after feeding [40]. Additionally, D4H transcript levels in seedlings were induced by JA only in the presence of light, whereas the expression of the upstream enzyme TDC was activated by JA even in the dark [26].

There are multiple layers of crosstalk between light and JA signaling. For example, PIFs activate a sulfotransferase that deactivates JA, leading to lower endogenous JA biosynthesis in the dark or shade [41]. However, this mechanism likely does not explain the light-dependent elicitation of D4H with exogenous JA. MYC2 Is also regulated by light. For example, it is post-translationally modified to be more active in blue light [43], degraded in the dark in a CONSTITUTIVELY PHOTOMORPHOGENIC1 (COP1)—dependent manner [42], and interacts with PIFs [44, 45], which may further contribute to its degradation in the dark [46]. The repression of MYC2 in the dark leads to attenuated JA-responsiveness in the dark. However, CrMYC2 does not appear to regulate vindoline pathway gene expression, even in the light [6, 14].

DELLAs can interact with over 300 different transcription factors, making them central regulators of numerous environmental inputs [47]. DELLAs are most known for their role as negative regulators of gibberellic acid (GA) signaling. In the presence of GA, DELLAs bind to GA-INSENSITIVE DWARF1 (GID1), which leads to the ubiquitination and degradation of DELLAs (reviewed in [48]). However, they are also important players in light and JA signaling (FIG. 1).

When seedlings are first exposed to light during de-etiolation, active GA levels decrease significantly, leading to a stabilization of DELLAs [49, 50]. The COP1 and SUPPRESSOR OF PHYA-105 1 (SPA1) complex can also bind to DELLAs in the dark or shade, ubiquitinating them and signaling for their degradation [51, 52]. In the light, DELLAs are stable and bind to PIFs, inhibiting PIF's ability to bind to DNA and contributing to their degradation [53-56]. Through these interactions, DELLAs are associated with enhanced light-activated photomorphogenesis in seedlings [57] and repressed shade avoidance responses in mature plants [58]. DELLAs can also bind and repress JAZs, amplifying JA responses. JAZs and PIFs compete for binding to DELLA, creating crosstalk between JA and light signaling [59]. In the light, PIFs are degraded, which frees DELLAs to bind and inhibit JAZs, amplifying JA responses in the light. When JA is present, JAZs are degraded, freeing DELLAs to bind and inhibit PIFs, which causes reduced growth in the presence of JA. Additionally, JA decreases active GA biosynthesis, increasing DELLA levels during pathogen attack [60].

### SUMMARY

Vinblastine (VBL) and vincristine (VCR) are two terpenoid indole alkaloids (TIAs) which are extracted from Catharanthus roseus for use as chemotherapy medicines. The present technology provides overexpression of transcription factors that activate multiple enzymes in the TIA biosynthetic pathway in order to increase VBL and VCR production. Upstream TIA pathway enzymes are highly activated by jasmonic acid (JA) and JA-responsive transcription factors. The downstream vindoline pathway, by contrast, is highly regulated by light and leaf-specific development. The central role in light and JA signaling of DELLA transcriptional activators make them prime targets for engineering increased expression of the JA-activated upstream TIA pathway and the light-activated vindoline pathway. In the present technology, DELLA transcriptional activators are used to integrate these two signals of defense and light to regulate both upstream and downstream TIA biosynthesis, leading to strong enhancement of the synthesis of vindoline and its subsequent products, VBL and VCR.

Two DELLA proteins, CrDELLA1 and CrDELLA2, were identified in C. roseus plants. Using a yeast-two hybrid assay, it was confirmed that CrDELLA1 can interact with JA-signaling JAZ proteins and light-signaling PIF proteins; JAZ and PIF proteins are repressors of the upstream TIA and vindoline pathways, respectively. When CrDELLA1 and CrDELLA2 were silenced together in C. roseus plants using virus induced gene silencing (VIGS), a constitutively shade-avoidant phenotype was observed, suggesting that CrDELLA1 and CrDELLA2 repress the activity of PIFs, activators of the shade-avoidant phenotype. CrDELLA silencing also led to a decrease in vindoline pathway gene expression, providing evidence that CrDELLAs positively regulate the vindoline pathway.

To Increase CrDELLA levels, CrGID1 was silenced or plants were treated with paclobutrazol (PAC). GID1s bind to DELLAs in the presence of gibberellic acid (GA), leading to the degradation of DELLAs, while PAC is an inhibitor of GA biosynthesis. Thus, DELLA protein levels can be increased by silencing CrGID1a and CrGID1b or by the addition of PAC, which lowers GA levels. A physical phenotype was observed in CrGID1-silenced plants that was the opposite of DELLA-silenced plants. In addition, the transient silencing CrGID1a/b increased vindoline pathway gene expression in older leaves, likely leading to increased levels of vindoline in mature leaves. Similarly, PAC treatment of etiolated seedlings increased several vindoline pathway genes. Thus, CrDELLA1 can activate both the upstream TIA pathway and the vindoline pathway, likely by binding and inhibiting JAZ and PIF activity. Construction of a gain-of-function DELLA mutant or a complete knockout of CrGID1a/b can be used to increase TIA and vindoline levels in C. roseus transgenic plants.

In one aspect, the technology provides a Catharanthus roseus (C. roseus) plant that contains one or more DELLA transcription factors which have enhanced activity compared that found in a naturally occurring C. roseus plant. The activity of one or more DELLA transcription factors can be enhanced (increased) compared to a naturally occurring C. roseus plant, or compared to a plant used as a starting point for enhancement, by at least 10%, at least 20%, at least 30%, at least 40%, at least 50%, at least 70%, at least 100%, at least 200%, at least 3-fold, at least 5-fold, at least 10-fold, or more. Enhancement of DELLA activity can be measured, for example, as any of the following: increase in expression of a DELLA gene, increase in intracellular DELLA protein concentration or amount, increase in binding of DELLA to a physiological target (such as another transcription factor known to bind DELLA under physiological conditions), or increase in binding affinity of a DELLA protein for a physiological target. As a result, the plant is capable of enhanced production of vindoline compared to a naturally occurring C. roseus plant. Naturally occurring C. roseus plants produce vindoline at a level of about 0.4-1.0 μg of vindoline/mg fresh wt of leaves. See Magnotta et al., Phytochemistry 67 2006) 1758-1764). Thus, enhanced production of vindoline compared to a naturally occurring C. roseus plant requires a higher level than this range, such as at least about 1.1, 1.2, 1.3, 1.5, 2, 2.5, or 3 or more μg of vindoline/mg fresh wt of leaves.

The plant can be produced by a number of different methods, but methods that produce stable genetic modifications in the plants genome are preferred. For example, the plant can be a transgenic plant or a plant developed by a selective breeding program. Short height is associated with enhanced DELLA activity and can be used as a selection factor. Alternatively, a molecular marker such as DELLA DNA, RNA, or protein sequence or intracellular level (concentration or amount, e.g., determined with a DELLA-specific antibody), or DELLA activity (e.g., binding to a target of DELLA, such as another transcription factor), or result of DELLA activity (e.g., level of a DELLA-controlled metabolite) can be used as a screening tool. Transgenic C. roseus plants can be prepared by introducing mutations into an endogenous DELLA gene, by substituting a more active DELLA gene from another species, or by increasing the DELLA gene copy number. A gain of function mutation is any genetic modification that results in an increased level or activity of DELLA under a given growth condition.

Another aspect of the technology is a method of preparing a C. roseus plant capable of enhanced vindoline, vinblastine, and/or vincristine production. The method includes Introducing a gain of function mutation in a CrDELLA1 gene and/or a CrDELLA2 gene into a C. roseus plant, and/or reducing the ability of a CrGID1a protein and/or a CrGID1b protein to cause degradation of CrDELLA1 protein and/or CrDELLA2 protein in the C. roseus plant. As a result, CrDELLA level and/or activity in the plant are enhanced, leading to enhanced production of vindoline, vinblastine, and/or vincristine by the plant.

Yet another aspect of the technology is a method of producing vinblastine and/or vincristine using a C. roseus plant. The method includes providing the C. roseus plant described above, having enhanced DELLA activity, and growing the plant under conditions suitable for the production of vinblastine and/or vincristine in the plant.

Still another aspect of the technology is an embodiment of the method of producing vinblastine and/or vincristine just described. The method further includes subjecting leaves of the plant to a treatment that enhances alkaloid biosynthesis in leaves of the plant, such as mechanically damaging the leaves, and waiting for a period of time, during which vindoline and catharanthine accumulate in the treated leaves. In some embodiments the method further includes harvesting the leaves and homogenizing the harvested leaves in a buffer solution, whereby said vindoline and catharanthine are released together with one or more enzymes involved in biosynthesis of vincristine and/or vinblastine. The homogenized leaves are then incubated, whereby vincristine and/or vinblastine are produced from reaction of said vindoline and catharanthine. This method overcomes the compartmentalization of certain precursors and enzymes in cells and tissues of the plant, and thereby accelerates vinblastine and vincristine synthesis; the method is described in published PCT Patent Application No. WO 2020/232412 A1.

Yet another aspect of the technology is a cell obtained from a C. roseus plant having enhanced DELLA activity, as described above. The cell can be isolated from the plant, such as from a transgenic C. roseus plant or a C. roseus plant that results from a selective breeding process. The cell can also be a C. roseus cell that has been engineered to include one or more genetic modifications present in a transgenic or selectively bred C. roseus plant having enhanced DELLA activity.

The present technology can be further summarized through the following list of features.

1. A Catharanthus roseus (C. roseus) plant comprising one or more DELLA transcription factors having enhanced activity compared to a naturally occurring C. roseus plant, wherein the plant is capable of enhanced production of vindoline compared to said naturally occurring C. roseus plant.

2. The plant of feature 1, wherein the plant is a transgenic plant or a plant obtained by selective breeding.

3. The plant of feature 1 or feature 2, wherein the DELLA transcription factor is obtained by mutation of a DELLA endogenous to C. roseus or is sourced from another species.

4. The plant of feature 1, wherein the plant has a higher intracellular level of CrDELLA1 and/or CrDELLA2 proteins than said naturally occurring C. roseus plant.

5. The plant of any of the preceding features, wherein the plant has a higher level of CrDELLA1 protein than said naturally occurring C. roseus plant.

6. The plant of any of the preceding features, wherein the plant has a higher level of CrDELLA2 protein than said naturally occurring C. roseus plant.

7. The plant of any of the preceding features, wherein one or more DELLA transcription factors activate a biosynthetic pathway leading to vindoline synthesis.

8. The plant of feature 7, wherein the one or more DELLA transcription factors activate a biosynthetic pathway from geraniol and tryptophan to tabersonine (i.e., terpenoid indole alkaloid (TIA) pathway) as well as a biosynthetic pathway from tabersonine to vindoline (vindoline pathway).

9. The plant of feature 7 or feature 8, wherein said one or more DELLA transcription factors bind and inhibit a jasmonate zim domain protein (JAZ) and/or a phytochrome interacting factor protein (PIF).

10. The plant of any of the preceding features, wherein the plant comprises a CrDELLA1 and/or CrDELLA2 gene harboring a gain of function mutation.

11. The plant of feature 10, wherein the gain of function mutation is selected from mutations that inhibit GID1a and/or GID1b binding to DELLA, mutations that inhibit degradation of DELLA in the presence of gibberellic acid, and mutations that disrupt DELLA-COP1 binding.

12. The plant of feature 11, wherein the gain of function mutation inhibits GID1a and/or GID1b from binding to DELLA, and wherein the mutation comprises an N-terminal deletion of up to 112 amino acids of CrDELLA1 and/or CrDELLA2.

13. The plant of any of the preceding features, wherein CrGID1a and/or CrGID1b protein intracellular levels are reduced in the plant.

14. The plant of feature 13, wherein the CrGID1a and/or CrGID1b genes are knocked down or knocked out in the plant.

15. The plant of any of the preceding features, wherein the plant is produced by a process comprising the use of CRISPR-Cas9, introduction of a mutated CrDELLA1, CrDELLA2, CrGID1a, and/or CrGID1b gene, introduction of one or more point mutations in a CrDELLA1, CrDELLA2, CrGID1a, and/or CrGID1b gene by random mutagenesis, or selective breeding.

16. The plant of any of the preceding features, wherein the plant is also capable of enhanced production of vinblastine and/or vincristine upon processing of leaves of the plant, compared to said naturally occurring C. roseus plant.

17. A method of preparing a C. roseus plant capable of enhanced vindoline production, the method comprising the steps of:
(a) introducing a gain of function mutation in a CrDELLA1 gene and/or a CrDELLA2 gene into a C. roseus plant; and/or
(b) reducing the ability of a CrGID1a protein and/or a CrGID1b protein to cause degradation of CrDELLA1 protein and/or CrDELLA2 protein in said C. roseus plant; whereby the C. roseus plant becomes capable of enhanced vindoline production.

18. The method of feature 17, whereby the C. roseus plant also becomes capable of enhanced production of vinblastine and/or vincristine upon processing of leaves of the plant.

19. The method of feature 17 or feature 18, wherein the gain of function mutation comprises deleting up to 112 amino acids at the N-terminus of CrDELLA1 and/or CrDELLA2.

20. The method of any of features 17-19, wherein point mutations in a CrDELLA1, CrDELLA2, CrGID1a, and/or CrGID1b gene are introduced by radiation or chemical agent mutagenesis or by selective breeding combined with screening for increased levels of CrDELLA1 protein and/or CrDELLA2 protein.

21. The method of any of features 17-20, wherein a transgenic C. roseus plant is produced.

22. A method of producing vinblastine and/or vincristine, the method comprising the steps of:
(a) providing the plant of any of features 1-16, or the plant made by a method comprising the method of any of features 17-21; and
(b) growing the plant under conditions suitable for the production of vindoline and catharanthine in the plant.

23. The method of feature 22, further comprising contacting the transgenic plant with a compound that reduces the level of gibberellic acid in the plant.

24. The method of feature 23, wherein the compound is selected from the group consisting of pacobutrazol cyclohexanetriones, ancymidol, tetcyclasis, and chloromequat chloride.

25. The method of any of features 22-24, further comprising activating a plant defense mechanism in the plant.

26. The method of feature 25, comprising contacting the plant with a plant defense hormone.

27. The method of feature 26, wherein the plant defense hormone is selected from the group consisting of jasmonate, methyl jasmonate, ethylene, ethephone, and 1-aminocyclopropane-1-carboxylic acid.

28. The method of any of features 22-27, further comprising exposing the plant to red light in a wavelength range of about 600-700 nm and blue light in a wavelength range of about 350-500 nm, wherein the red light and blue light are of increased intensity relative to other wavelengths of a solar spectrum.

29. The method of any of features 22-28, further comprising the steps of:
(c) subjecting leaves of the plant to a treatment that enhances alkaloid biosynthesis in leaves of the plant; and
(d) waiting for a period of time, during which vindoline and catharanthine accumulate in said leaves.

30. The method of feature 29, further comprising the steps of
(e) harvesting said leaves;
(f) homogenizing the harvested leaves in a buffer solution, whereby said vindoline and catharanthine are released from cells of the harvested leaves and one or more enzymes involved in biosynthesis of vincristine and/or vinblastine are also released from cells of the harvested leaves; and
(g) incubating the homogenized leaves, whereby vincristine and/or vinblastine are produced from reaction of said vindoline and catharanthine.

31. A cell obtained from the plant of any of features 1-16, or a C. roseus cell bearing identical genetic modifications compared to said plant.

### BRIEF DESCRIPTION OF THE DRAWINGS

FIG. 1A shows a diagram depicting the integration of light and JA signaling in the regulation of TIA biosynthesis. Dotted lines indicate transcriptional regulation while solid lines represent post-translational regulation (sequestration or degradation). All interactions have been previously characterized in A. thaliana or C. roseus. Jasmonic acid (JA) biosynthesis is induced by necrotrophic pathogen attack or herbivory. JA signals the degradation of JAZ proteins [3]. JAZs bind and repress CrMYC2 [4], which transcriptionally activates upstream TIA pathway genes and other activators of TIA biosynthesis, ORCAs and BISs [4, 5, 14, 6-13]. In response to light, PhyA and PhyB dissociate the COP1/SPA1 complex, inactivating it [61, 62]. In light, PhyA and PhyB also bind to PIFs, inhibiting their activity and signaling their degradation [21]. CrPIF1 transcriptionally represses the vindoline pathway and CrGATA1, an activator of vindoline biosynthesis [20]. DELLAs bind and repress the activity of JAZs and PIFs [53-56, 59]. JAZs also inhibit DELLAs' ability to bind to other transcription factors like PIFs [59]. In the dark, the COP1/SPA1 complex binds to DELLAs and signals for their degradation [51, 52]. DELLAs are also degraded in the presence of gibberellic acid (GA) [48]. Active GA synthesis is transcriptionally repressed by JA [60], and by PhyA in the light [49, 50]. FIG. 1B shows a biosynthetic pathway leading to the production of vinblastine and vincristine in C. roseus.

FIGS. 2A and 2B show that CrDELLA1Δ1-209 can interact with CrJAZ1 and CrPIF4/5 in a yeast-two hybrid assay. FIG. 2A shows growth on SC-L-T-H+50 mM media, which indicates a positive protein-protein interaction. FIG. 2B shows that growth on SC-L-T serves as a positive control. SC-L-T-H: Synthetic complete media lacking leucine, tryptophan, and histidine with 50 mM 3-aminotriazole (3AT). SC-L-T: Synthetic complete media lacking leucine and tryptophan. For each interaction, three colonies were screened (columns) in three dilutions (rows—from top to bottom: 2×, 10×, 100×). CrDELLA1Δ1-209 with empty pDEST™22 was tested with only a 10× and 100× dilution.

FIGS. 3A-3G show that silencing CrDELLA1 and CrDELLA2 leads to an elongated, hyponastic phenotype, similar to a constitutively active shade avoidance response. (3A-3C) The second leaf pair that emerged after infection are slender in DELLA-silenced plants compared to GFP-silenced plants (a greater length:width ratio) and have a longer petiole. (3D,3E) After the point of infection, DELLA-silenced plants exhibit a slightly elongated stem compared to GFP-silenced plants. (3F,3G) DELLA-silenced plants had a hyponastic leaf angle compared to GFP-silenced plants. Each data point is from one plant whose silencing was confirmed with qPCR. Leaf measurements are an average of both leaves in a leaf pair. Apical stem length and leaf length:width ratio were measured in three experimental repeats and combined for analysis (38, 3E). Plants that had a leaf pair die after pinching were removed from the analysis of stem length due to changes in apical dominance. Petiole length and leaf angle were measured in two experimental repeats (3C, 3G). Boxes represent the 25^{th} and 75^{th} percentile with a line marking the median. Whiskers extend to the minimum and maximum. ** p<0.01, * p<0.05, two-tailed t-test.

FIGS. 4A-4D show that DELLA silencing led to significant decreases in some vindoline pathway genes (T3O, T3R, NMT, D4H) (4A), but this decrease seems highly dependent on the developmental status of the leaf. Little effect was seen on the more mature, first leaf pair after infection (4B). No decreases were observed in the second experimental repeat (4C) and a decrease in only a single gene, DAT, was observed in the third experimental repeat (4D). Each replicate is a single leaf from an individual plant (N=5). Relative gene expression was measured with qPCR and calculated using the 2^{-ΔΔCt} method [77] relative to the control condition (GFP-silenced plants) of the respective leaf pair, and normalized relative to the housekeeping gene, SAND [76]. Boxes represent the 25^{th} and 75^{th} percentile with a line marking the median. Whiskers extend to the minimum and maximum. *p<0.05, **p<0.01, ***p<0.001, ****p<0.0001, two-tailed t-test.

FIGS. 5A and 5B show that silencing CrGID1a and CrGID1b led to small but insignificant increases in vindoline pathway gene expression in the first leaf pair (5B) that emerged after infection but had no effect in the second leaf pair (5A). Each replicate is a single leaf from an individual plant (N=5). Relative gene expression was measured with qPCR and calculated using the 2^{-ΔΔCt} method [77] relative to the control condition (GFP-silenced plants) of the respective leaf pair, and normalized relative to the housekeeping gene, SAND [76]. Boxes represent the 25^{th} and 75^{th} percentile with a line marking the median. Whiskers extend to the minimum and maximum. *p<0.05, **p<0.01, ***p<0.001, ****p<0.0001, two-tailed t-test.

FIGS. 6A and 6B show that application of PAC to etiolated seedlings leads to a moderate increase in expression of some vindoline pathway genes. Application of 1 μM PAC to etiolated seedlings led to a significant decrease in seedling height compared to the mock treatment (DMSO). Height was measured using ImageJ. (6B) PAC treatment led to a significant increase in LHCB2.2 (positive control) and 16OMT gene expression. However, it decreased expression of D4H, DELLA1, and DELLA2. Non-significant increases in other vindoline pathway genes (T3O, T3R, NMT, and DAT) was also observed. For gene expression, each replicate (N=5) is a pool of 3 whole seedlings. Relative gene expression was measured with qPCR and calculated using the 2^{-ΔΔCt} method [77] relative to the control condition (Mock treatment), and normalized relative to the housekeeping gene, SAND [76]. Boxes represent the 25^{th} and 75^{th} percentile with a line marking the median. Whiskers extend to the minimum and maximum. *p<0.05, **p<0.01, two-tailed t-test with an FDR cutoff of 20% according to the two-stage step-up method of Benjamini, Krieger and Yekutieli.

FIG. 7 shows an amino acid sequence alignment of DELLA proteins. Protein sequences were downloaded from Uniprot with accession numbers: AtRGA (Arabidopsis thaliana, O.9SLH3—SEQ ID NO:1); AtGAI (Arabldopsis thafana, O.9LO.T8—SEQ ID NO:2); AtRGL1 (Arabidopsis thaliana, O.9C8Y3—SEQ ID NO:3); AtRGL2 (Arabidopsis thaliana, O.8GXW1-SEQ ID NO:4); AtRGL3 (Arabidopsis thaliana, O.9LF53—SEQ ID NO:5); AtSCR (Arabidopsis thaliana, O.9M384—SEQ ID NO:6, outgroup); OsSLR1 (Oryza sativa, O.7G7J6—SEQ ID NO:7); ZmD8 (Zea mays, O.9ST48—SEQ ID NO:8); ZmD9 (Zea mays, O.06F07—SEQ ID NO:9); SIGAI (Solanum lycopersicum, O.7Y1B6—SEQ ID NO:10); TaRht-D1 (Triticum aestivum, O.9ST59—SEQ ID NO:11); HvSLN1 (Hordeum vulgare, O.8W127—SEQ ID NO:12); PsLA (Pisum sativum, B2BA72—SEQ ID NO:13); PsCRY (Pisum sativum, B2BA71-SEQ ID NO:14); VvGAI1 (Vitis viniftera, O.8S4W7—SEQ ID NO:15); BrRGA1 (Bassica rapa, O.5BN23—SEQ ID NO:16); BrRGA2 (Bassica rapa, O.5BN22—SEQ ID NO:17). Sequences were aligned with CrDELLA1 (CRO_T106013—SEQ ID NO:18), CrDELLA2 (CRO_T106004—SEQ ID NO:19), and the two next closest homologues in C. roseus, CRO_T119352 (SEQ ID NO:20) and CRO_T119350 (SEQ ID NO:21), using CLC Main Workbench 21.0.3 (gap open cost=15.0, gap extension cost=1.0). Lines indicate conserved domains, and filled boxes indicate defining amino acid residues for each domain. Annotations are defined according to Itoh 2002 [64] and Pysh 1999 [65].

FIG. 8 shows an amino acid sequence alignment of GID1 proteins. Protein sequences were downloaded from UniProt: OsGID1 (Oryza sativa, Q6L545—SEQ ID NO:22), AtGID1A (Arabidopsis thaliana, Q9MAA7—SEQ ID NO:23), AtGID1B (Arabidopsis thaliana, Q9LYC1—SEQ ID NO:24), AtGID1c (Arabidopsis thaliana, Q940G6—SEQ ID NO:25), AtHSL1 (Arabidopsis thaliana, Q9LT1—SEQ ID NO:26). Sequences were aligned with CrGID1a (CRO_T105824—SEQ ID NO:27), CrGID1b (CRO_T119046—SEQ ID NO:28), and the next closest homologue in C. roseus, CRO_T115705 (SEQ ID NO:29), using CLC Main Workbench 21.0.3 (default parameters: gap open cost=10.0, gap extension cost=1.0). Open circles are conserved residues important for binding to DELLA Filled circles are the “catalytic triad”—SDH in HSLs, and SDV/I present in GID1s. Annotations are defined according to [66].

FIGS. 9A-9F show that silencing CrGID1a and CrGID1b (CrGID1a/b) leads to a shortened, epinastic phenotype, opposite to the phenotype observed with CrDELLA silencing. (9A-9B) After the point of infection, CrGIDa/b-silenced plants exhibit a slightly shortened stem compared to GFP-silenced plants. (9C-9D) CrGID1a/b-silenced plants had an epinastic leaf angle compared to GFP-silenced plants. (9E-9F) The two pairs of leaves that emerged after infection are short and wide (a lower length:width ratio) in CrGIDa/b-silenced plants compared to GFP-silenced plants. Each data point is from one plant whose silencing was confirmed with qPCR, except for plants used for stem measurements, which were not confirmed with qPCR. Leaf measurements are an average of both leaves in a leaf pair. Boxes represent the 25^{th} and 75^{th} percentile with a line marking the median. Whiskers extend to the minimum and maximum. All measurements are from one experimental repeat. * p<0.05, two-tailed unpaired t-test.

FIG. 10 shows leaves from CrDELLAs-silenced and CrGID1-silenced plants compared to GFP-silenced plants (negative control) In “Experiment 2” (leaves were not individually photographed for “Experiment 1”).

FIG. 11 shows that CrDELLA1 and CrDELLA2 are expressed more in immature leaves than mature leaves, similar to the vindoline pathway. Each replicate is a single leaf from an individual plant (N=5). Relative gene expression was measured with qPCR and calculated using the 2^{-ΔΔCt} method [77] relative to the control condition (GFP-silenced plants) of the first leaf pair after VIGS infection, and normalized relative to the housekeeping gene, SAND [76]. Boxes represent the 25^{th} and 75^{th} percentile with a line marking the median. Whiskers extend to the minimum and maximum. *p<0.05, **p<0.01, ***p<0.001, two-tailed t-test.

FIG. 12 shows that silencing CrDELLA1 did not impact TDC and G10H Expression. Results are from “Experiment 1” (see FIGS. 4A, 4B). Each replicate is a single leaf from an individual plant (N=5). Relative gene expression was measured with qPCR and calculated using the 2^{-ΔΔCt} method [77] relative to the control condition (GFP-silenced plants) of the respective leaf pair, and normalized relative to the housekeeping gene, SAND [76]. Boxes represent the 25^{th} and 75^{th} percentile with a line marking the median. Whiskers extend to the minimum and maximum. No statistical significance was found with a two-tailed t-test.

### DETAILED DESCRIPTION

The present technology provides transgenic plants in which the action of DELLA transcription factors are used to coordinate light and defense signals and thereby to activate TIA synthesis and the vindoline synthetic pathway. In particular, two DELLA proteins are identified in C. roseus plants, one of which, CrDELLA1, can be utilized as a positive regulator of vindoline synthesis.

DELLAs are transcription factors from the GRAS protein family, named after the three genes that define this family in Arabidopsis thaliana: GIBBERELLIN-ACID INSENSITIVE (GAJ), REPRESSOR of GAI (RGA), and SCARECROW (SCR). All GRAS proteins contain a conserved C-terminal domain which is responsible for many protein-protein interactions; the C-terminal domain consists of two leucine heptad repeats (LHRI and LHRII) and three other motifs (VHIID, PFYRE, and SAW) [64, 65]. Specifically, the central VHIID domain is necessary for interaction with most proteins, including JAZs and PIFs [54, 79-81]; the PFYRE and SAW domains are not necessary for interaction with JAZs [82] and PIFs [54] but are required for interaction with other proteins like IDDs [79, 83] and ARRs [80]. The LHRI domain is necessary for homodimerization of DELLAs [64].

DELLAs like GAI and RGA are distinguished from SCR and SCR-Like (SCL) proteins by their conserved N-terminus, which contains the DELLA and TVHYNP motifs. Specifically, the DELLA and TVHYNP motifs contain transactivation activity [94]. In addition, the N-terminal domain is necessary for binding to GID1 In the presence of gibberelic acid, leading to DELLA's degradation [84, 85]. Mutations and deletions in the N-terminus are responsible for gain-of-function mutations by stabilizing DELLAs [85-93]. N-terminal deletions of CrDELLA1 and/or CrDELLA2, produced by deleting an N-terminal portion ranging in length from the first 50 to the first 220 amino acids, or the first 112 to 209 amino acids, or the first 50, 60, 70, 80, 90, 100, 110, 112, 115, 120, 150, 175, 200, 209, or 220 amino acids, yield gain of function mutations by preventing the binding of GID1 in the presence of gibberellic acid. The C-terminus also contributes to the stability of GID1 binding, although it is not necessary for binding [87].

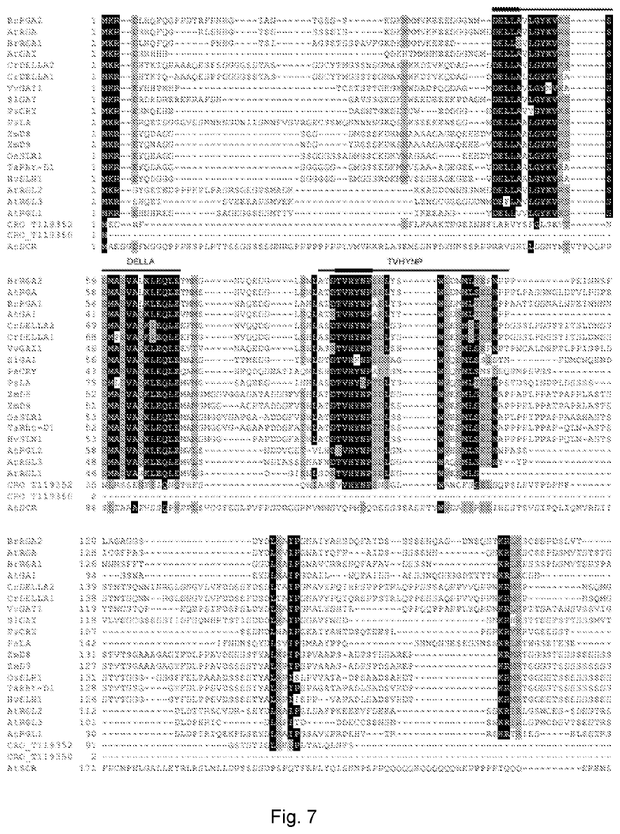

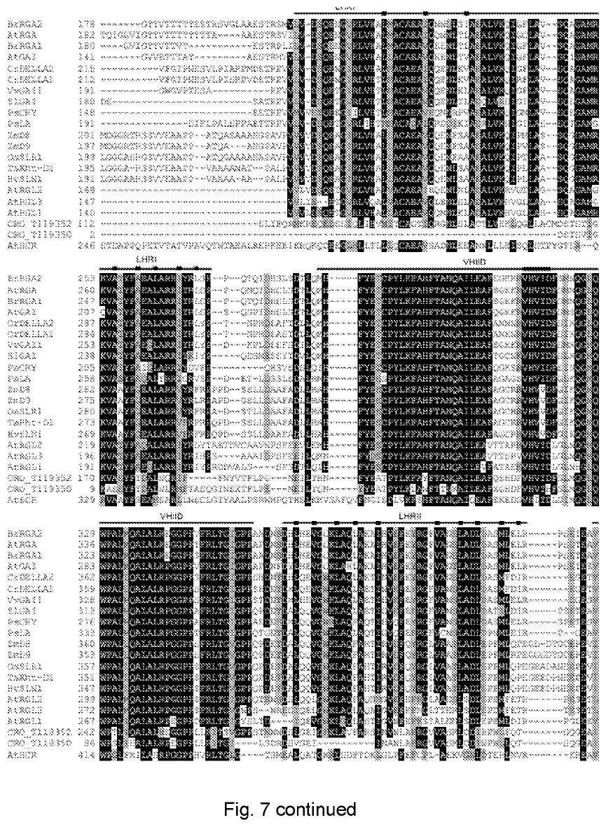

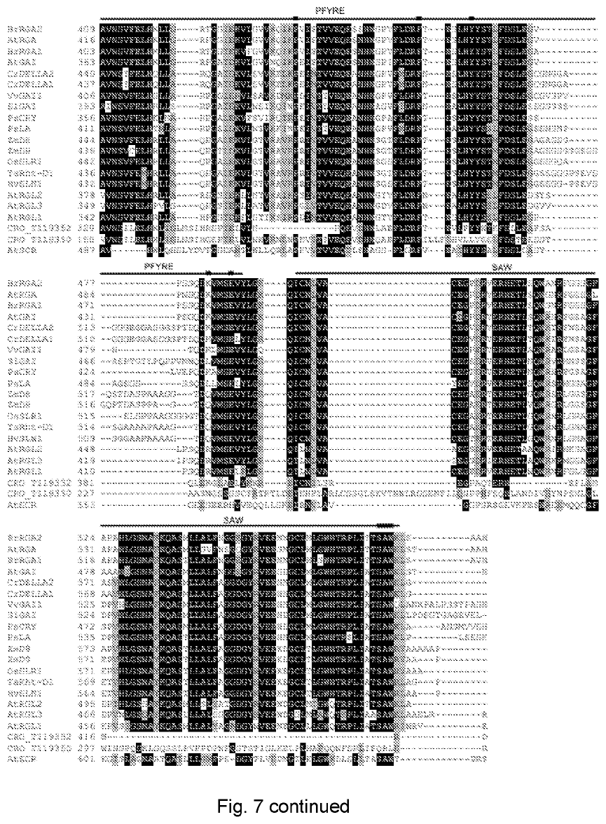

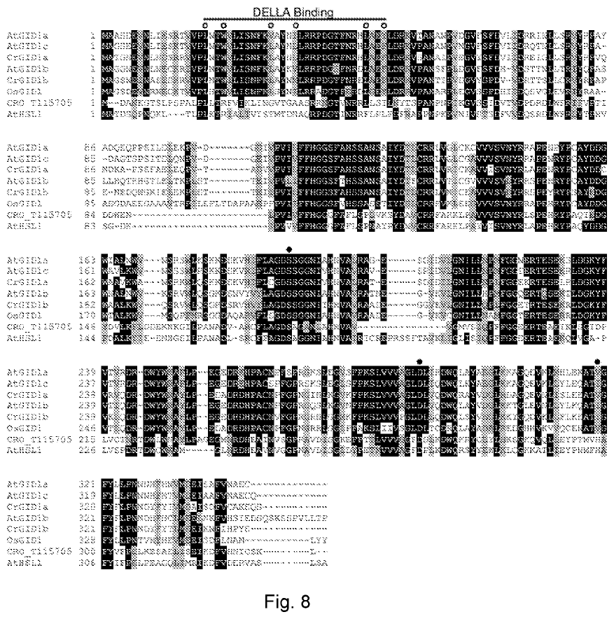

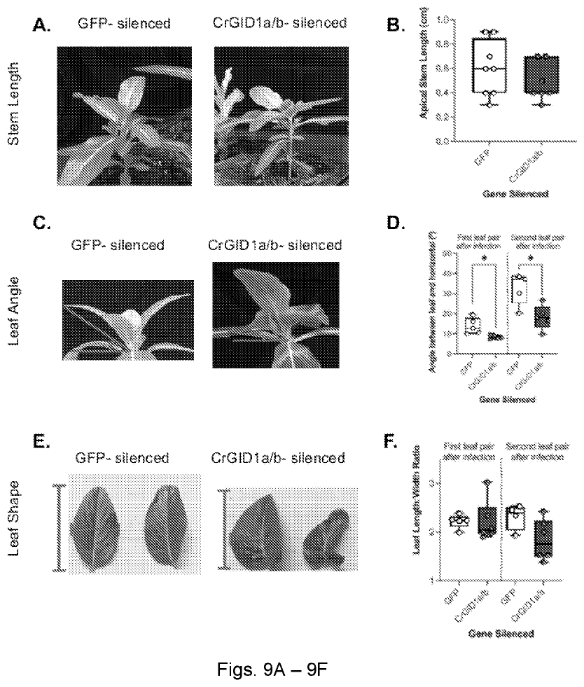

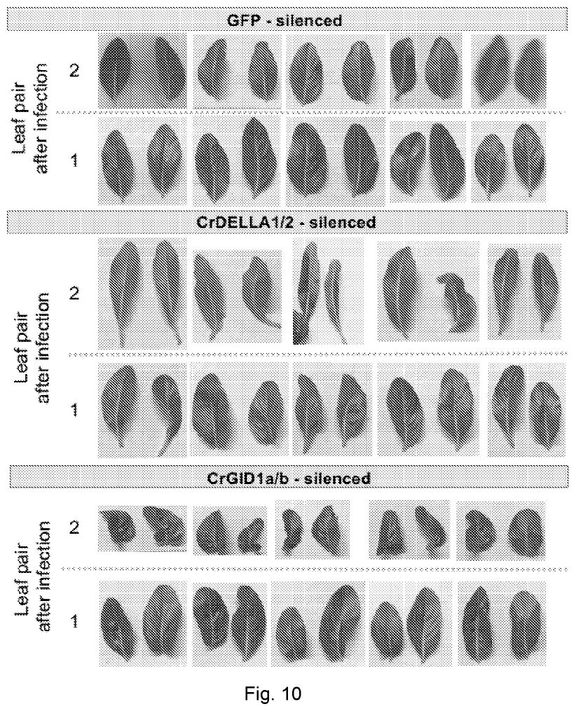

Arising from a single ancestor in bryophytes, DELLA genes have been duplicated and lost numerous times throughout tracheophyte evolution, leading to a variability in the number of DELLA genes between species [56, 95]. For example, A. thaliana has 5 DELLA genes (GAI, RGA, RGA-LIKE1 (RGL1), RGL2, and RGL3); rice and tomato only have one DELLA gene [56]; and pea has two DELLA genes [96]. To determine how many DELLA genes are expressed in the C. roseus genome, a BLASTP search was performed using the DELLA domain sequence from the A. thaliana RGA1 protein as a query against the C. roseus version 2 translated transcriptome [97]. Two sequences (CRO_T106013 (CrDELLA1) and CRO_T106004 (CrDELLA2)) were returned with very low e-values (less than 1E-29). An amino acid alignment with characterized DELLA proteins from other species (FIG. 7) confirmed the presence of the N-terminal DELLA and the TVHYNP domains and the C-terminal leucine heptad repeats (LHRI and LHRII), VHIID, PFYRE, and SAW motifs [64, 65]. The next closest hits from the BLASTP search, CRO_T119352 and CRO_T119350 (with e-values less than 5E-4), were included in the alignment to confirm that these sequences did not have the conserved domains present in DELLA proteins.

CrDELLA1 and CrDELLA2 are clustered on the same genomic scaffold (cro_v2_scaffold_123). Using available C. roseus transcriptome data [15], it was found that CrDELLA2 Is most highly expressed in mature leaves while CrDELLA1 Is most highly expressed in stems. This is different from expression patterns of vindoline pathway genes, which are most highly expressed in young leaves, but DELLAs undergo significant post-translational regulation [47] so it is unclear how much these expression levels correlate with active protein levels.

The CrDELLA1 and CrDELLA2 genes from C. roseus var. Little Bright Eye cDNA were amplified and cloned, confirming that they were expressed and that the coding sequences predicted by CRO_T106013 and CRO_T106004 are correct.

To confirm the role of CrDELLAs in mediating light and defense signaling in C. roseus, a yeast-two hybrid (Y2H) assay was used to investigate whether CrDELLA1 can interact with CrJAZ or CrPIF proteins, similar to its interaction and repression of JAZs and PIFs in other species. See FIGS. 2A-2B. In C. roseus, there are five characterized JAZ proteins that bind to CrMYC2, repressing the transcription of important TIA regulator and biosynthetic genes, ORCA3, BIS1, and G10H [4, 98]. CrDELLA1 's interaction with CrJAZ1 was tested as a representative CrJAZ protein. In C. roseus, there are three identified PIF proteins [99]. CrDELLA1's interaction with CrPIF4/5 was tested as a representative CrPIF protein.

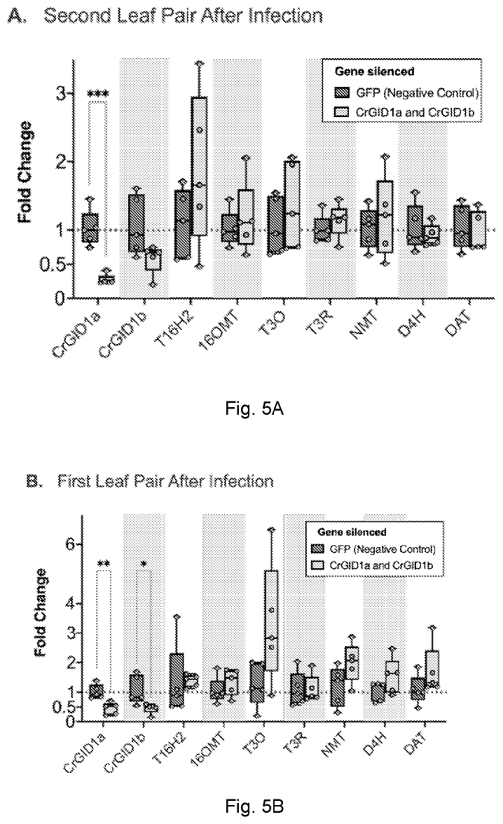

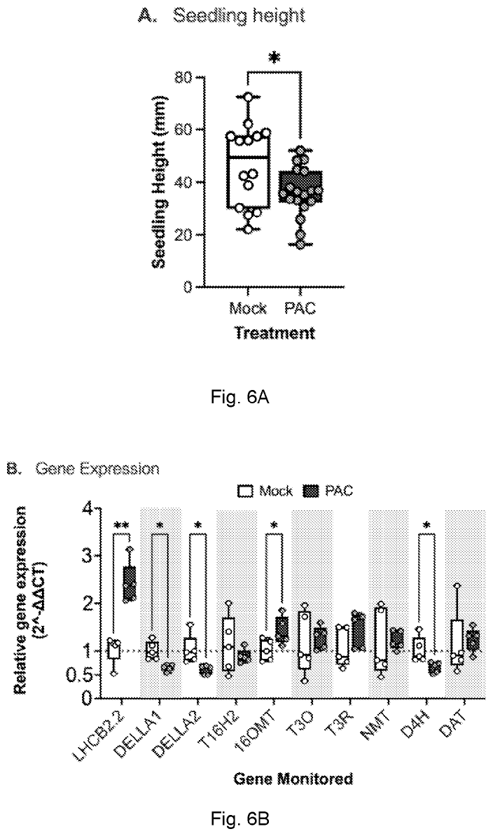

DELLAs and PIFs both contain activation domains, leading to false positive results in Y2H assays when used as the bait (fused to the DNA binding domain) [94]. To avoid self-activation, the DELLA and TVHYNP motifs in the N-terminus of CrDELLA1 (amino acids 1-209: CrDELLA1Δ1-209) were removed. A similar truncation in A. thaliana was shown to remove self-activation but retain interaction with JAZs and PIFs in Y2H assays [54, 82]. CrDELLA1Δ1-209 was cloned into the pDEST™32 bait plasmid containing the GAL4 DNA binding domain while CrJAZ1Δ1-84 and CrPIF4/5 were cloned into the pDEST™22 prey plasmid containing the GAL4 activation domain. Yeast containing CrDELLA1Δ1-209 and CrJAZ1Δ1-84 or CrPIF4/5 were able to grow on media lacking histidine, indicating that CrDELLA1Δ1-209 was able to interact with CrJAZ1 and CrPIF4/5 (FIG. 5.2). These results confirm that CrDELLA1 acts like DELLAs in other species by interacting with defense-signaling JAZ proteins and light-signaling PIF proteins.

To explore the function of CrDELLA1 and CrDELLA2 in vivo, both DELLAs were silenced simultaneously using virus-induced gene silencing, which leads to reduced gene expression in the two leaf pairs that emerge after viral infection.

In A. thaliana, quadruple or quintuple DELLA knockouts lead to phenotypes similar to the shade avoidance response (SAS). In response to low light, plants tend to elongate their hypocotyls and petioles, bend their leaves upward (hyponasty), reduce branching, and accelerate flowering. Collectively, these physical changes are termed the shade avoidance response [100]. In DELLA knockout mutants, Djakovic-Petrovic et al. observed constitutively elongated hypocotyls but not petioles [58] and Küpers et al. observed hyponasty [101]. These phenotypes suggest that DELLAs are partially responsible for repressing SAS, likely through their interaction and repression of PIFs.

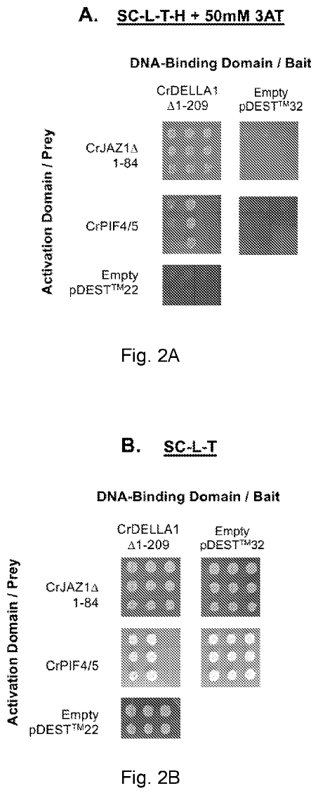

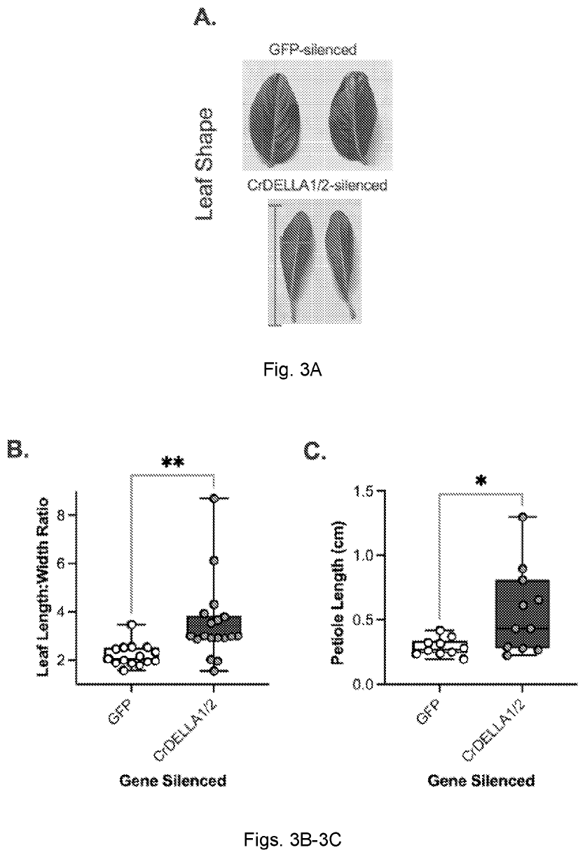

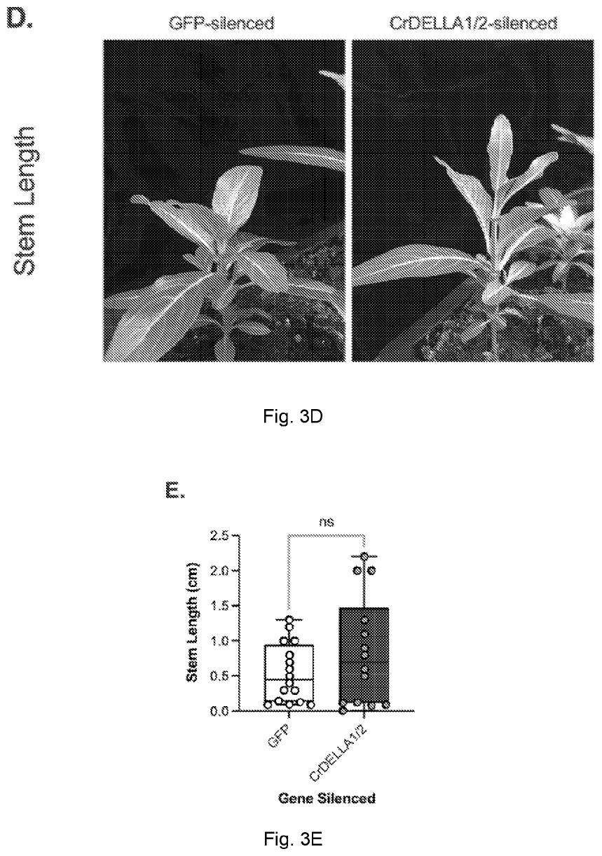

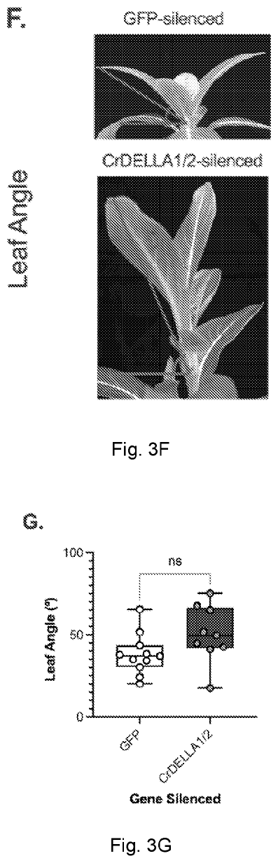

When CrDELLA1 and CrDELLA2 were silenced together (CrDELLA1/2), a visible and quantifiable phenotype compared to the GFP-silenced negative control was observed. In addition to elongated stems and a hyponastic leaf angle observed in A thaliana DELLA knockouts, it was also found that silencing of CrDELLA1 and CrDELLA2 led to elongated leaves and petioles (FIGS. 3A-3G). This phenotype is also similar to a previously observed phenotype of C. roseus (increased stem and leaf lengths) with addition of GA, which would result in DELLA degradation [102]. These physical changes confirm that CrDELLA1 and CrDELLA2 play a similar role as AtDELLAs in repressing SAS.

As a complementary experiment, CrGID1a and CrGID1b genes were also identified and silenced simultaneously. GID1 binds to DELLAs in the presence of GA, signaling the degradation of DELLAs. Thus, silencing GID1 in C. roseus should lead to elevated protein levels of CrDELLA1 and CrDELLA2. Two putative GID1 genes were identified in C. roseus that are homologous to A thaliana GID1s and that contain conserved amino acid residues responsible for binding to DELLAs: CrGID1a (CRO_T105824) and CrGID1b (CRO_T119046) (FIG. S5.2) [66].

The physical phenotype of CrGID1-silenced plants was less prominent than the CrDELLA1/2-silenced phenotype, but the plants did show slightly shortened stems and leaves and a slightly epinastic leaf angle compared to GFP-silenced plants. As expected, the GID-silenced phenotype was opposite to the DELLA-silenced phenotype (FIGS. 3A-3G).

Thus, silencing of CrDELLA1 and CrDELLA2 leads to an elongated constitutive shade-avoidance phenotype, while silencing of CrGID1 leads to a disrupted SAS phenotype.

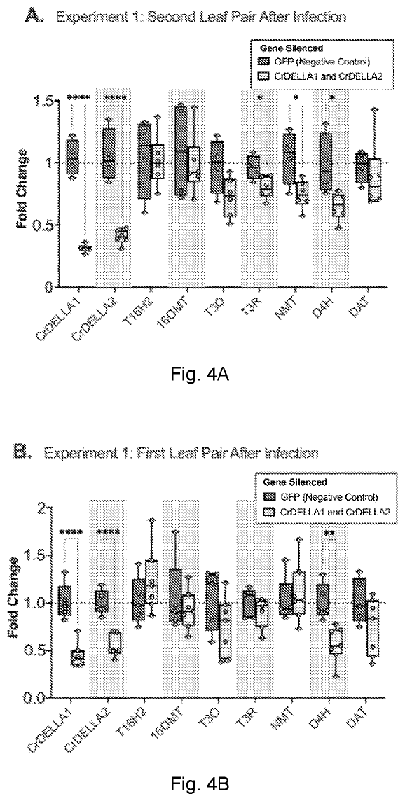

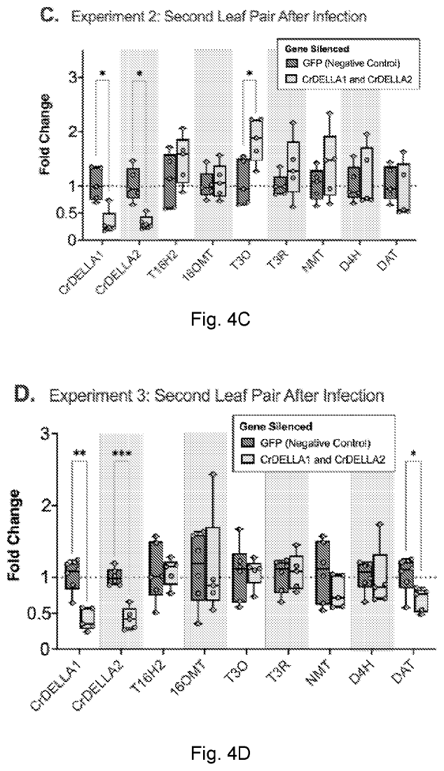

Next investigated was how silencing CrDELLA1 and CrDELLA2 affected vindoline pathway gene expression. RNA was extracted from a single leaf from the first or second leaf pair that emerged after infection, and gene expression was monitored with qPCR. Both CrDELLA1 and CrDELLA2 were successfully silenced, with an approximately 50% decrease in expression in the first leaf pair after infection (FIG. 4B) and a >50% decrease in expression in the second leaf pair after infection (FIG. 4A). It was additionally noticed that expression of both CrDELLA1 and CrDELLA2 were higher in the younger leaves (2^{nd} leaf pair after infection) compared to the more mature leaves (1^{st} leaf pair after infection), similar to vindoline pathway genes (FIGS. 5A-5B). This contrasted with previous RNAseq data that found higher expression of CrDELLA2 in mature leaves [15], but the RNAseq data are from only one biological replicate while the present qPCR data are from 5 replicates.

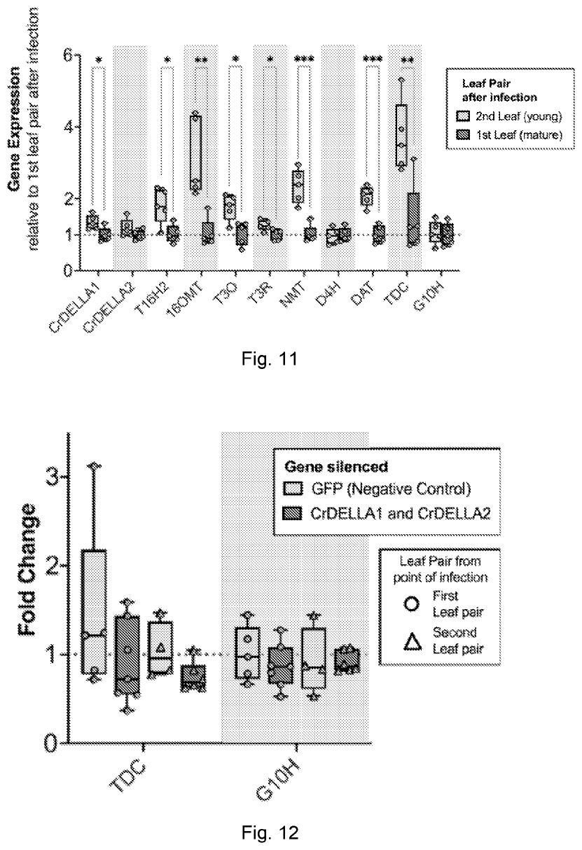

In the younger second leaf pair after infection, silencing of CrDELLA1 and CrDELLA2 led to moderate (20-35%) but significant decreases in the expression of T3R, NMT, and D4H (FIG. 4A). Only D4H showed decreased expression in the older first leaf pair after infection. In contrast to the vindoline pathway, upstream TIA genes, TDC and G10H were not affected by DELLA silencing (FIG. 12). ORCA3 and STR transcript levels were also monitored but were too low to accurately quantify.

The different effect observed in the two leaf pairs could be due to stronger silencing in the younger second leaf pair, higher basal expression of vindoline pathway genes in the second leaf pair (FIG. 11), or some other developmental factor influencing vindoline pathway gene expression. Interestingly, D4H is the only vindoline pathway gene that did not show significantly higher basal expression in the younger leaf pair (FIG. 11) and is the only gene that showed an effect in the older leaf pair, suggesting that some developmental factor that does not influence D4H may be responsible for the muted effect of DELLA silencing in the older leaf pair.

This experiment was repeated two more times with only the second leaf pair after infection (younger leaf) analyzed. Although CrDELLA1 and CrDELLA2 were successfully silenced in both of these experiments, there was only a slight decrease in DAT expression in Experiment 3 (FIG. 4C).

CrGID1a and CrGID1b were also successfully silenced (FIGS. 5A-5B). Although we did not observe statistically significant increases in vindoline pathway gene expression, there were non-significant increases in all vindoline pathway genes, consistent with the hypothesis that DELLAs activate the vindoline pathway. Specifically, silencing CrGID1a and CrGID1b by about 50% led to a 78% increase on average in vindoline pathway gene expression, in the first leaf pair after infection. Silencing GID1a/b in the second leaf pair after infection led to a 24% increase on average in vindoline pathway gene expression. These results suggest that CrDELLA1 and CrDELLA2 are important for vindoline pathway gene expression under specific developmental contexts.

To further validate the role of DELLAs in activating the vindoline pathway genes during photomorphogenesis, seedlings were treated with paciobutrazol (PAC), a chemical that inhibits gibberellic acid (GA) synthesis. In the presence of GA, GID1 binds to DELLA proteins and signals their degradation. Thus, the inhibition of GA synthesis should lead to an increase in DELLA protein levels. However, it has also been shown that the interaction between GID1 and DELLA is disrupted in the presence of blue light [103, 104], so PAC treatment likely won't have an effect on DELLA protein levels in the light where DELLA is already stabilized. This is what is seen by Alabadi et al. and Cheminant et al., who observed increases in photosynthesis-related genes when PAC was applied to seedlings in the dark but not in the light [105, 106]. Similar to how PAC increases photosynthesis-associated genes in the dark by disrupting DELLA degradation, it was investigated whether PAC treatment would be able to activate vindoline pathway gene expression in the dark.

Because PAC might disrupt germination, seedlings were first germinated normally without any treatment by incubating them in the dark for 5 days at 27° C. The germinated seeds were then transferred to either media containing 1 μM PAC or media containing an equal volume of DMSO as a mock treatment. These seedlings were kept in the dark for 4 more days before being harvested for gene expression analysis.

A significant decrease in seedling height was observed in seedlings that were treated with PAC. This is consistent with GA's positive role in growth and served as a good indicator that the PAC was having an effect on the seedlings. As a positive control, expression of CrLHCB2.2 was measured; CrLHCB2.2 is a homologue of the light harvesting complex subunit B 2.2, which was previously shown to be activated by PAC treatment of dark-grown seedlings in A. thaliana [106]. A significant >2-fold activation of CrLHCB2.2 was observed with PAC treatment.

When vindoline pathway gene expression was monitored, approximately 50% activation was seen for 5 of the 7 vindoline pathway genes (16OMT, T3O, T3R, NMT, and DAT), although only 16OMT showed a statistically significant increase. Surprisingly, D4H showed a significant decrease with PAC treatment, while T16H2 showed no effect.

The effect PAC had on DELLA transcript levels was also checked. Both CrDELLA1 and CrDELLA2 had significantly decreased expression with PAC treatment. Since PAC is supposed to stabilize DELLA protein levels, it is likely that this decrease in gene expression is a negative feedback reaction to increased protein levels.

These results indicate that stabilization of DELLAs with PAC treatment can increase expression of the vindoline pathway.

The amino acid sequences of CrDELLA1 and 2 as well as CrGID1a and 1b are published at the following accession numbers, the content of which is hereby incorporated by reference: CRO_T106013 (CrDELLA1), CRO_T106004 (CrDELLA2), CRO_T105824 (CrGID1a), CRO_T119046 (CrGID1b).

### EXAMPLES

### Example 1. Identification of CrDELLA1, CrDELLA2, CrGID1a, And CrGID1b

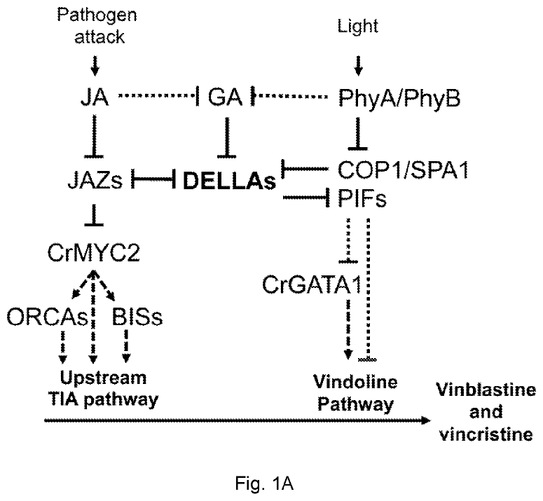

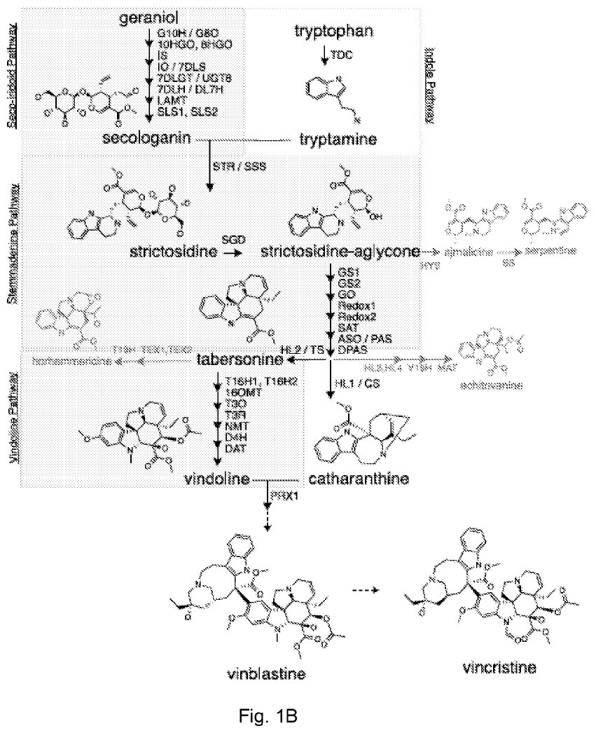

To identify DELLA genes in C. roseus, the N-terminal DELLA and TVHYNP domains from the A thaliana RGA protein (DDELLAVLGYKVRSSEMAEVALKLEQLETMMSNVQEDGLSHLATDTVHYNPSELYSWLDN MLSELNPPPLP, SEQ ID NO:30) was used in a Protein Basic Alignment Search Tool (BLASTP) search in the C. roseus v.2 translated transcriptome with default parameters (BLOSUM62 Matrix; Gap cost existence=11; Gap cost extension=1) [63]. The two putative DELLA sequences identified by this search (CrDELLA1=CRO_T106013 and CrDELLA2=CRO_T106004), and the two next closest homologues in C. roseus (CRO_T119352 and CRO_T119350) were aligned against DELLA and GRAS proteins from other species using CLC Main Workbench 21.0.3 (gap open cost=15.0, gap extension cost=1.0). Amino acid sequences used in the alignment were downloaded from UniProt: AtRGA (Arabidopsis thaliana, Q9SLH3); AtGAI (Arabidopsis thaliana, Q9LQT8); AtRGL1 (Arabidopsis thaliana, Q9C8Y3); AtRGL2 (Arabidopsis thaliana, Q8GXW1); AtRGL3 (Arabidopsis thaliana,Q9LF53) AtSCR (Arabidopsis thaliana, Q9M384, outgroup); OsSLR1 (Oryza saliva, Q7G7J6); ZmD8 (Zea mays, Q9ST48); ZmD9 (Zea mays, Q06F07); SIGAI (Solanum lycopersicum, Q7Y1B6); TaRht-D1 (Triticum aestivum, Q9ST59); HvSLN1 (Hordeum vulgare, Q8W127); PsLA (Pisum sativum, B2BA72); PsCRY (Pisum sativum, B2BA71); VvGAI1 (Vitis vinifera, Q8S4W7); BrRGA1 (Brassica rapa, Q5BN23); BrRGA2 (Brassica rapa, Q5BN22). Domain annotations on the amino acid alignment are defined according to Itoh et al. 2002 [64] and Pysh et al. 1999 [65]. The results are shown in FIG. 1.

To identify GID1 genes in C. roseus, we performed a BLASTP search with the three A thaliana GID1 amino acid sequences (AtGID1A—Q9MAA7; AtGID1B—Q9LYC1, AtGID1c—Q940G6). The two putative GID1 sequences identified by this search, CrGID1a (CRO_T105824) and CrGID1b (CRO_T119046), and the next closest homologue, CRO_T115705, were aligned against O. saliva and A. thaliana GID1 proteins using CLC Main Workbench 21.0.3 (default parameters: gap open cost=10.0, gap extension cost=1.0). Amino acid sequences used in the alignment were downloaded from UniProt OsGID1 (Oryza saliva, Q6L545), AtGID1A (Arabidopsis thaliana, Q9MAA7), AtGID1B (Arabidopsis thaliana, Q9LYC1), AtGID1c (Arabidopsis thaliana, Q940G6), AtHSL1 (Arabidopsis thaliana, Q9LT10). Annotations on the amino acid alignment are defined according to Gazara at al. 2018 [66]. The results are shown in FIG. 8.

### Example 2. Cloning of CrDELLA1 and CrDELLA2

Coding sequences for CrDELLA1 (CRO_T106013), CrDELLA2 (CRO_T106004), CrPIF4/5 (KR703668.1, CRO_T136917)[20], and CrJAZ1Δ1-84 (FJ040204.1, CRO_T107113) [4, 67] were amplified from C. roseus var. Little Bright Eye cDNA using Phusion High-Fidelity DNA Polymerase (New England BioLabs) and Gateway-compatible primers. CrDELLA1 and CrDELLA2 were amplified from cDNA prepared from a pool of C. roseus tissue types (fruits, flower buds, flowers, stems, leaves, and roots). The expected band size for each CDS was cut out of an agarose gel and purified using the Zymoclean Gel DNA Recovery kk (Zymo Research). PCR products were cloned into the entry plasmid pDONR™221 using the Gateway® BP Clonase™ II Enzyme Mix and then cloned into the Yeast-2-Hybrid Gateway prey vector, pDEST™22, or bait vector, pDEST™32, using the Gateway® LR Clonase™ II Enzyme Mix. To obtain the truncated CrDELLA1Δ1-209 in the pDEST™32 bait plasmid, round-the-horn PCR was used on the entry vector, pDONR™221-CrDELLA1, with primers targeting amino acids 1 to 209.

For silencing experiments, 300 bp fragments targeting CrDELLA1, CrDELLA2, CrGID1a, and CrGID1b were designed using the Sol Genomics Network (SGN) VIGS tool [68](n-mer=21-23, mismatches=1), which minimized off-targets in the C. roseus version two transcriptome [63]. Fragments were amplified from C. roseus leaf cDNA (CrGID1a and CrGID1b) or from previously cloned cDNA (CrDELLA1 and CrDELLA2) and cloned into pTRV2-GG (Addgene plasmid #105349) using modular cloning. Plasmids targeting CHLH and GFP for silencing were cloned previously.

To overexpress CrDELLA1, CrDELLA2, or CrDELLA1Δ1-209, the coding sequences were amplified from previously cloned plasmids using Golden-Gate compatible primers. The amplified PCR products were gel extracted, cut with BpiI and ligated into a level zero backbone. CrDELLA1 CDS was ligated into pICH41308 (Level zero CDS1 backbone); CrDELLA2 and CrDELLA1Δ1-209 were ligated into pAGM1287 (Level zero CDS1ns backbone). Stop codons weren't included in the amplification for ease of C-terminal tagging. Coding sequences were cloned into a transcriptional unit in the pICH47732 Level 1 Forward position 1 vector backbone, containing the Cauliflower mosaic virus 2×35S promoter, Tobacco Mosaic Virus omega 5′UTR, Agrobacterium tumefaciens MAS terminator, and stop codons for coding sequences. To facilitate cloning of the stop codon, an additional serine and glycine was added to the C-terminal end of the coding sequences. This transcriptional unit was moved into the pSB90 backbone (Addgene plasmid #123187), which includes the right and left borders required for Agrobacterium-mediated transfer into plant cells, and a mutated VirG gene to enhance Agrobacterium virulence [69]. As a negative control, pSB161 [69], containing Beta-glucuronidase (GUS) with an intron under control of the 2×35S promoter was used. To overexpress CrDELLA1Δ434-621 or CrDELLA1Δ1-112, the Q5® Site-Directed Mutagenesis Kit was used to delete the nucleotides that coded for the respective amino acids (434-621 or 1-112) from the level 2 plasmid overexpressing CrDELLA1.

Reporter plasmids containing vindoline pathway, ORCA3, and STR promoters were cloned previously [69, 70]. All primers used for cloning are listed in Table 1. Sequences were confirmed after every PCR amplification using Sanger Sequencing at Genewiz®. Final plasmids were confirmed with a restriction enzyme digest and visualization with agarose gel electrophoresis. All plasmids were electroporated into Agrobacterium tumefaciens GV3101 (pMP90).

TABLE 1
Primers. Uppercase indicates sequences complementary to its target while lowercase
indicates 5′ overhangs to facilitate cloning.

| Primer Name | Primer Sequence | Purpose |
|---|---|---|
| DELLAFattb1_2 | ggggacaagtttgtacaaaaaagcaggctggAT | Amplification of |
| | GAAGAGGGACCATACG | CrDELLA1 and CrDELLA2 |
| DELLARattb2 | ggggaccactttgtacaagaaagctgggtagtTT | for Y2H Assay |
| | AACCGAGTTTCCAG | (Gateway compatible) |
| PIF4Fattb1_2 | ggggacaagtttgtacaaaaaagcaggctctAT | Amplification of |
| | GTATCCTTGCTTTCCTG | CrPIF4/5 for Y2H Assay |
| PIF4Rattb2 | ggggaccactttgtacaagaaagctgggtactTC | (Gateway compatible) |
| | AGCCTAGACTTTGC | |
| JAZ1Fattb1_2 | ggggacaagtttgtacaaaaaagcaggctcgAT | Amplification of |
| | GGATAAATCTGGCCAG | CrJAZ1Δ1-84 for Y2H |
| JAZ1Rattb2 | ggggaccactttgtacaagaaagctgggtgggT | Assay |
| | TTTAAAAAGGAAAGCC | (Gateway compatible) |
| LC19_DELLA1_F | aagaagacaaaATGAAGAGGGACCATAC | Amplification of |
| | GAAAAC | CrDELLA1 for |
| LC20_DELLA1_R | aagaagacaaaagcTTAACCGAGTTTCCA | overexpression |
| | GGCCG | (MoClo compatible) |
| LC57_DELLA2F | aagaagacaaaATGAAGAGGGACCATAC | Amplification of |
| | G | CrDELLA2 for |
| LC58_DELLA2R | aagaagacaacgaaccACCGAGTTTCCAG | overexpression |
| | GC | (MoClo compatible) |
| LC19_DELLA1_F | aagaagacaaaATGAAGAGGGACCATAC | Amplification of |
| | GAAAAC | CrDELLA1 for cloning |
| LC58_DELLA2R | aagaagacaacgaaccACCGAGTTTCCAG | with additional 2 AAs |
| | GC | |
| LC59_DELLA1_V1_F | tttggtctcatatgACCCTTTTCGATTCACT | Amplification of |
| | TG | CrDELLA1 fragment for |
| LC60_DELLA1_V1_R | tttggtctcaatttCACTCTATAGCCATCTC | VIGS silencing |
| | CAC | |
| LC61_DELLA2_V1_F | tttggtctctAAATTCGCTCACTTTACAGC | Amplification of |
| | | CrDELLA2 fragment for |
| LC62_DELLA2_V1_R | tttggtctcacaccTGCATCAAGATCAGCA | VIGS silencing |
| | AG | |
| LC82_GID1a_VIGS_F | tttggtctcatatgCTATTTGTATGATATCA | Amplification of CrGID1a |
| | AGATTCG | fragment for VIGS |
| LC83_GID1a_VIGS_R | tttggtctcaggcaATGCCAACTTGAAATT | silencing |
| | TGAG | |
| LC84_GID1b_VIGS_F | tttggtctctTGCCGTAGATTAGTTAACA | Amplification of |
| | | CrGID1b fragment for |
| LC85_GID1b_VIGS_R | tttggtctcacaccTGATTCTGTTCTTGTTT | VIGS silencing |
| | CTC | |
| LC72_DELLA1_Ctrunc_ | GCCTTCACGAATATCAAAC | Cloning of |
| F | | CrDELLA1Δ434-621 |
| LC73_DELLA1_Ctrunc_ | TAAGCTTGGACTCCCATG | (via Q5 directed site |
| R | | mutagenesis) |
| LC95_DELLA1_del1_ | TGTATCGATAATTGTAAATGTAATTG | Cloning of |
| 112_F | | CrDELLA1Δ1-112 |
| LC96_DELLA1 del1_ | ATGATTTCTGAAATCAACCC | (via Q5 directed site |
| 112_R | | mutagenesis) |
| LC63_DELLA1_Trunc_ | aagaagacaaaATGGGCGTTTTTGGGA | Amplification and |
| F | | cloning of CrDELLA1Δ1- |
| LC58_DELLA2R | aagaagacaacgaaccACCGAGTTTCCAG | 209 |
| | GC | |
| RH_DELLA_trunc1_F | ATGGGCGTTTTTGGGATTCCAAATGA | N-terminal truncation of |
| | GTCTG | CrDELLA1 for Y2H |
| RH_DELLA_trunc1_R | CCAGCCTGCTTTTTTGTACAAAGTTGG | (via round-the-horn |
| | | PCR) |
| DELLA1_qF | GCTGAGGCTATCCAACAAGAA | qPCR amplifying |
| DELLA1_qR | GCCAATGCTTCAGCGAAATAG | CrDELLA1 |
| DELLA2_qF | GGTATGGCAAAGATGTGGAAAG | qPCR amplifying |
| DELLA2_qR | CAGCCATGTCCGAGGATTTA | CrDELLA2 |
| GID1a_qF | TTGCTGGGCTGCTGTAAA | qPCR amplifying |
| GID1a_qR | CCACCAGAACTATCACCACATAA | CrGID1a |
| GID1b_qF | CTAACTTCAAGCTGGCCTACA | qPCR amplifying |
| GID1b_qR | GAATTGTGTTAGCAGGGACTTTC | CrGID1b |
| q2T16h_up | GATCAACTCACAGTGGCAGTC | qPCR amplifying T16H2 |
| q2T16h_down | GACTTGAGACTTGTGATTGGC | |
| 16OMTvigs-qF1 | GTGTGAAGATACTCAAAAGCTGC | qPCR amplifying 16OMT |
| 16OMTvigs-qR1 | CAAAATTTACAAGCATTGCCATATCC | |
| T3O_qF | GTCATAGACGAGCACAGAGAAA | qPCR amplifying T3O |
| T3O_qR | CACCACCCTCTTCAATCCTAAG | |
| T3R_qF | CTTGAGCCACTCTTTGCTTTAC | qPCR amplifying T3R |
| T3R_qR | ATGAGGGACATTGCGGATAC | |
| 227O-qF1 | TGACAAAGTAACCGGAGCATGGGA | qPCR amplifying NMT |
| 227O-qR1 | ATCCGAATGACGGCATCTTGGCTA | |
| D4Hvigs-QF1 | TGGCCTCAGTAGCAATTCAG | qPCR amplifying D4H |
| D4Hvigs-QR1 | TCCATATTTCTCACTCGCTTCTC | |
| DATvigs-qF1 | GAGGTTTTGACTGCTTTTCTCAG | qPCR amplifying DAT |
| DATvigs-qR1 | TGGAAATGGCAAAGATTGGC | |
| SAND_qF | TGCTGTGGAGGAGGAAGAAG | qPCR amplifying SAND |
| SAND_qR | ACTGGCGGAACTACTACTACC | |

### Example 3. Yeast Two-Hybrid Assay

Yeast-Two-Hybrid (Y2H) assays were conducted using the ProQuest™ Two-Hybrid System from Invitrogen. CrJAZ1Δ1-84 and CrPIF4/5 coding sequences were cloned into the pDEST™22 backbone containing the GAL4 Activation Domain (prey), and CrDELLA1Δ1-209 was cloned into the pDEST™32 backbone containing the GAL4 DNA binding domain (bait). Yeast strain MaV203 was co-transformed with a prey and bait plasmid using the LiAc/SS carrier DNA/PEG method [71] and plated on synthetic complete (SC) media lacking leucine (to select for pDEST™32), and tryptophan (to select for pDEST™22) (SC-L-T: 27 g/L dropout base medium (MP Biomedicals), 1.57 g/L synthetic complete medium-His-Leu-Trp-Ura (Sunrise Science Products), 100 mg/L adenine hemisulfate, 85.6 mg/L histidine, 85.6 mg/L uracil, 173.4 mg/L leucine, 20 g/L agar, pH adjusted to 5.8-5.9).

Three colonies for each transformation were screened for positive interaction by plating on SC-L-T-H selection media containing 50 mM 3-amino-1,2,4-triazole (3-AT) added after autoclaving following the dilution series method described by Cuéllar et al. [72].

### Example 4. Virus-Induced Gene Silencing

C. roseus var. Little Bright Eye (NESeeds, 0.4 g) were sterilized by submersion in 70% ethanol for 45 seconds, 30% bleach and 1× Triton for 6 minutes, triple-rinsed in sterile water, and incubated in 3% Plant Preservative Mixture (PPM) for 18 hours in the dark; the PPM was decanted, and the seeds were spread on full-strength Gamborg's media (3.1 g/L Gamborg's basal salts, 1× Gamborg's vitamins, and 6% micropropagation agar type 1, Phytotechnology Laboratory) inside a sterile Magenta™ Plant Culture Box (Sigma) for germination. Seeds were germinated in the dark at 25-27° C. until seedlings were about 2 cm tall (about 7 days). Seedlings were then transferred to 16 hr light/8 hr dark photoperiods (red and blue LED lights, about 80 μmol m⁻² s⁻¹) for at least two days. Once seedlings had undergone photomorphogenesis, they were planted in soil (Miracle-Gro) in 2.25′×2.25′ cels and grown under the same 16 hr light/8 hr dark photoperiod (red and blue LED lights, about 90 μmol m⁻² s⁻¹) until two true leaves appeared (about 4-6 weeks).

Once seedlings had two true leaves, they were infected with Agrobacterium tumefaciens according to the pinch-wounding method [73]. A single colony of A. tumefaciens GV3101 (pMP90) harboring pTRV1 or pTRV2 was used to inoculate a 10 mL culture of LB with Gentamycin (10 mg/L, selects for pMP90) and Kanamycin (50 mg/L, selects for pTRV1 and pTRV2-GATEWAY) or Spectinomycin (100 mg/L, selects for pTRV2-GG) in a 50 mL conical centrifuge tube. This culture was grown at 26° C. and 250RPM for two days. It was then pelleted, resuspended in Induction media (10.46 g/L Agrobacterium minimal medium (PlantMedia), 100 μM acetosyringone) with antibiotics, and grown for another 3 hours. It was then pelleted again and resuspended in 1 mL of infiltration media without Silwet® L-77 (10 mM MgSO4, 10 mM MES pH 5.8, 200 μM acetosyringone). A. tumefaciens strains containing pTRV2 and pTRV1 plasmids were combined in a 1:1 ratio (OD600 of each strain=2-4). Modified tweezers were dipped into the A. tumefaciens solution and the plant was pinched three times in the highest internode beneath the shoot apical meristem (dipping into the solution between each pinch).

After infection, plants were kept in the dark for two days before being placed back into a 16 hour light/8 hour dark photoperiod under red and blue LED lights (about 90 μmol m⁻² s⁻¹). Light measurements are an average of five measurements taken with the Apogee SQ-520 Full Spectrum Smart Quantum Sensor.

Plants were grown until two pairs of leaves emerged after silencing and the CHLH-silenced plant exhibited yellow leaves (about 2-3 weeks). At this point, a single leaf from the two youngest leaf pairs were individually harvested for RNA extraction.

### Example 5. Plant Phenotype Measurements

To phenotype DELLA and GID1 silenced plants, stem lengths were measured with a ruler from the point of infection to the shoot apical meristem. Any plants whose leaves died immediately after infection were removed from the stem length analysis as this injury led to reduced apical dominance and influenced the length of the main stem.

Leaf lengths and widths were measured with a ruler for two experimental repeats. For one experimental repeat, the leaves were laid flat next to a ruler and photographed. Lengths and widths were measured from photographs using ImageJ. Lengths were measured from the base of the petiole to the tip of the leaf. Widths were measured at the widest part of the leaf. All petiole lengths were measured using ImageJ.

For leaf angle measurements, plants were photographed perpendicular to the angle of each of the leaf pairs. ImageJ was used to calculate the angle between the leaf lamina and the plane normal to the stem (equivalent to the horizontal plane [41, 74]). Only the second leaf pairs that emerged after infection were included in leaf length, width, and angle measurements.

Seedlings treated with PAC were photographed and lengths were measured with ImageJ. Statistical significance for all phenotype measurements was analyzed with a two-tailed unpaired T-test.

### Example 6. RNA Extraction and Quantitative PCR

Gene expression levels were monitored using quantitative real-time PCR (qRT-PCR) with primers listed in Table 1. mRNA was extracted from liquid nitrogen flash-frozen leaf tissue or seedlings stored at −80° C. While still frozen, tissue was crushed by shaking in a Mini-BeadBeater-16 (Biospec) for 15 seconds with ten 3 mm glass beads (Fisher). Afterwards, RNA was extracted with RNAzol-RT (Molecular Research Center) and the Direct-zol RNA Miniprep Plus Kit (Zymo Research) with on-column DNAse treatment to remove genomic DNA. RNA integrity was assessed using agarose gel electrophoresis, and concentration and purity were quantified with a NanoDrop (ND-1000 Spectrophotometer; ThermoScientific). cDNA was synthesized using either the SuperScript II First-Strand Synthesis System (Invitrogen) or the LunaScript RT SuperMix Kit (New England Biolabs) with up to 2.5 μg of RNA, according to manufacturer's instructions. cDNA was diluted 1:4, and 1 μL was used in a 10 μL reaction with SYBR Green ROX qPCR Master Mix (Qiagen or ABClonal) and 300 nM primers on the MX3000P qPCR instrument (Agilent) using the thermocycler protocol previously described with an extension time of 30 seconds [75]. Ct values for each biological replicate were calculated as the average of two technical replicates. Transcript levels were normalized to the housekeeping gene, SAND [76], and fold changes relative to the negative control condition were calculated according to the 2a method [77]. Amplification efficiency for each primer set was confirmed using Ct values over a range of cDNA dilutions and was 100%±10% for each gene monitored. Specificity of the primers were confirmed by gel electrophoresis and sequencing. SAND Ct values in no reverse-transcriptase controls were confirmed to be at least 5 Ct values above the respective experimental sample, indicating minimal genomic DNA contamination [78].

### Example 7. Paclobutrazol Treatment of Seedlings

C. roseus var. Little Bright Eye seeds (NESeeds, 0.8 g) were sterilized by submersion in 4% Plant Preservative Mixture (PPM) for 18 hours in the dark. The PPM was then decanted, and the seeds were spread on full-strength Gamborg's media (3.1 g/L Gamborg's basal salts, 1× Gamborg's vitamins, and 6% micropropagation agar type 1, Phytotechnology Laboratory) inside a sterile Magenta™ Plant Culture Box (Sigma) for germination. Seeds were germinated in the dark at 27° C. for 5 days until the radicle of the seedling had just emerged. A 100 mM stock solution of paclobutrazol (PAC, PhytoTechnology Laboratories) was prepared with DMSO as the solvent, filter-sterilized, and stored at −20° C. until use. A final concentration of 1 μM PAC or an equivalent amount of DMSO (mock) was added to Gamborg's media after autoclaving. After germination, seedlings were sterilely transferred to a new Magenta™ Box containing Gamborg's media containing 1 μM PAC or DMSO. Seedlings were maintained in the dark at 27° C. for 4 days, and then were harvested by placing 3 whole seedlings in a 2 mL screw cap tube containing ten 3 mm glass beads for each biological replicate, flash-freezing in liquid nitrogen, and storing at −80° C. until ready for RNA extraction and qPCR analysis.

### Example 8. Production of a Catharanthus roseus Transgenic Plant

The Catharanthus roseus transgenic plant is developed by first introducing the foreign DNA construct (transcriptional unit encoding and expressing the gene of interest [117,118], silencing fragment [119], CRISPR-Cas9 [120], etc.) into a single Catharanthus roseus cell and then regenerating an entire plant (shoots plus roots) from that transformed single cel; all cells of the resulting plant will contain the foreign DNA cassette [121]. In the first stage of the development of the transgenic plant, individual protoplasts, individual cells, individual cells within a given tissue (within leaf explants, [122], or within apical meristem [123]) are transformed with the DNA construct using either particle bombardment, disarmed Agrobacterium tumefaciens (Rhizobium radiobacter), or Agrobacterium rhizogenes (Rhizobium rhizogenes). The transformed cells are selected from the untransformed cells through a selection marker (like an antibiotic resistance gene or a gene involved in the biosynthesis of a necessary nutrient). In the second stage of the development of the transgenic plant, the transformed cells are placed on media containing specific combinations of plant hormones to induce either root or shoot formation. Then, the tissue is placed on a different combination of plant hormones to induce either the complementary tissue, i.e. shoot or root, completing the entire plant.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein.

Published PCT Patent Application No. WO 2020/232412 A1 is hereby incorporated by reference in is entirety.

### REFERENCES

1. Kulagina N, Méteignier L-V, Papon N, O'Connor S E, Courdavault V (2022) More than a Catharanthus plant: A multicellular and pluri-organele alkaloid-producing factory. Curr Opin Plant Biol 67:102200. doi.org/10.1016/j.pbi.2022.102200
2. Kazan K, Manners J M (2013) MYC2: The Master in Action. Mol Plant 6:686-703. doi.org/doi.org/10.1093/mp/sss128
3. Wasternack C, Hause B (2013) Jasmonates: biosynthesis, perception, signal transduction and action in plant stress response, growth and development. An update to the 2007 review in Annals of Botany. Ann Bot 111:1021-1058. doi.org/10.1093/aob/mt067
4. Patra B, Pattanaik S, Schluttenhofer C, Yuan L (2018) A network of jasmonate-responsive bHLH factors modulate monoterpenoid indole alkaloid biosynthesis in Catharanthus roseus. New Phytol 217:1566-1581. doi.org/10.1111/nph.14910
5. Zhang H, Hedhili S, Montiel G, Zhang Y, Chatel G, Pré M, Gantet P, Memelink J (2011) The basic helix-loop-helix transcription factor CrMYC2 controls the jasmonate-responsive expression of the ORCA genes that regulate alkaloid biosynthesis in Catharanthus roseus. Plant J 67:61-71. doi.org/10.1111/j.1365-313X.2011.04575.x
6. Schweizer F, Colinas M, Pollier J, Van Moerkercke A, Vanden Bossche R, de Clercq R, Goossens A (2018) An engineered combinatorial module of transcription factors boosts production of monoterpenoid indole alkaloids in Catharanthus roseus. Metab Eng 48:150-162. doi.org/10.1016/j.ymben.2018.05.016
7. van der Fits L, Memelink J (2000) ORCA3, a Jasmonate-Responsive Transcriptional Regulator of Plant Primary and Secondary Metabolism. Science (80-) 295:295-297. doi.org/10.1126/science.289.5477.295
8. Menke F L H, Champion A, Kljne J W, Memelink J (1999) A novel jasmonate- and elicor-responsive element in the periwinkle secondary metabolite biosynthetic gene Str interacts with a jasmonate- and elicitor-inducible AP2—domain transcription factor, ORCA2. EMBO J 18:4455-4483. doi.org/10.1093/emboj/18.16.4455
9. Van Moerkercke A, Steensma P, Gariboldi I, Espoz J, Pumama P C, Schweizer F, Miettinen K, Vanden Bossche R, De Clercq R, Memelink J, Goossens A (2016) The basic helix-loop-helix transcription factor BIS2 is essential for monoterpenoid indole alkaloid production in the medicinal plant Catharanthus roseus. Plant J 3-12. doi.org/10.1111/tpj.13230
10. Van Moerkercke A, Steensma P, Schweizer F, Pollier J, Gariboldi I, Payne R, Vanden Bossche R, Miettinen K, Espoz J, Pumama PC, Kellner F, Seppänen-Laakso T, O'Connor S E, Rischer H, Memelink J, Goossens A (2015) The bHLH transcription factor BIS1 controls the iridold branch of the monoterpenoid indole alkaloid pathway in Catharanthus roseus. Proc Natl Acad Sci 112:8130-8135. doi.org/10.1073/pnas.1504951112
11. Singh SK, Patra B, Paul P, Liu Y, Pattanaik S, Yuan L (2021) BHLH IRIDOID SYNTHESIS 3 is a member of a bHLH gene cluster regulating terpenoid indole alkaloid biosynthesis in Catharanthus roseus. Plant Direct 5:00305. doi.org/doi.org/10.1002/pld3.305
12. Singh S K, Patra B, Paul P, Liu Y, Pattanaik S, Yuan L (2020) Revisiting the ORCA gene cluster that regulates terpenoid indole alkaloid biosynthesis in Catharanthus roseus. Plant Sd 293:110408. doi.org/10.1016/j.plantsci.2020.110408
13. Paul P, Singh S K, Patra B, Sul X, Pattanaik S, Yuan L (2017) A differentially regulated AP2/ERF transcription factor gene cluster acts downstream of a MAP kinase cascade to modulate terpenoid indole alkaloid biosynthesis in Catharanthus roseus. New Phytol 213:1107-1123. doi.org/10.1111/nph.14252
14. Colinas M, Polier J, Vaneechoutte D, Malat D G, Schweizer F, De Milde L, De Clercq R, Guedes J G, Martinez-Cortés T, Molina-Hidalgo F J, Sottomayor M, Vandepoele K, Goossens A (2021) Subfunctionalization of Paralog Transcription Factors Contributes to Regulation of Alkaloid Pathway Branch Choice in Catharanthus roseus. Front Plant Sci 12:687406. doi.org/10.3389/fpls.2021.687406
15. Góngora-Castillo E, Childs K L, Fedewa G, Hamilton J P, Liscombe D K, Magalanes-Lundback M, Mandadi K K, Nims E, Runguphan W, Vallancourt B, Varbanova-Herde M, DelaPenna D, McKnight T D, O'Connor S, Buell C R (2012) Development of Transcriptomic Resources for Interrogating the Biosynthesis of Monoterpene Indole Alkaloids in Medicinal Plant Species. PLoS One 7: doi.org/10.1371/journal.pone.0052506
16. Besseau S, Kelner F, Lanoue A, Thamm A M K, Salim V, Schneider B, Geu-Flores F, Hofer R, Gulrimand G, Gulhur A, Oudhi A, Glevarec G, Foureau E, Papon N, Clastre M, Giglioli-Guivarc'h N, St-Pierre B, Werck-Reichhart D, Burlat V, De Luca V, O'Connor S E, Courdavault V (2013) A Pair of Tabersonine 16-Hydroxylases Initiates the Synthesis of Vindoline in an Organ-Dependent Manner in Catharanthus roseus. Plant Physiol 163:1792-1803. doi.org/10.1104/pp.113.222828
17. Vazquez-Flota F A, De Luca V (1998) Developmental and light regulation of desacetoxyvindoline 4-hydroxylase in Catharanthus roseus (L.) G. Don. Evidence Of a multilevel regulatory mechanism. Plant Physiol 117:1351-61
18. Yu B, Liu Y, Pan Y, Liu J, Wang H, Tang Z (2018) Light enhanced the biosynthesis of terpenoid indole alkaloids to meet the opening of cotyledons in process of photomorphogenesis of Catharanthus roseus. Plant Growth Regul 0:1-10. doi.org/10.1007/s10725-017-0366-0
19. Unterbusch E, Kalenbach M, Strack D, Luca V De, Schro J (1999) Light-induced cytochrome P450-dependent enzyme in indole akaloid biosynthesis: tabersonine 16-hydroxylase. Fed Eur Biochem Soc 458:97-102
20. Liu Y, Patra B, Pattanaik S, Wang Y, Yuan L (2019) GATA and Phytochrome Interacting Factor Transcription Factors Regulate Light-Induced Vindoline Biosynthesis in Catharanthus roseus. Plant Physiol 180:1336-1350. doi.org/10.1104/pp.19.00489
21. Legris M, Ince Y Q, Fankhauser C (2019) Molecular mechanisms underlying phytochrome-controlled morphogenesis in plants. Nat Commun 10:5219. doi.org/10.1038/s41467-019-13045-0
22. Li J, U G, Wang H, Wang Deng X (2011) Phytochrome signaling mechanisms. Arab B 9:e0148-e0148. doi.org/10.1199/tab.0148
23. Liscombe D K, Usera A R, O'Connor S E (2010) Homolog of tocopherol C methyltransferases catalyzes N methylation in anticancer alkaloid biosynthesis. Proc Natl Acad Sci 107:18793-18798. doi.org/10.1073/pnas.1009003107
24. Wei S (2010) Methyl jasmonic acid induced expression pattern of terpenoid indole alkaloid pathway genes in Catharanthus roseus seedlings. Plant Growth Regul 61:243-251. doi.org/10.1007/s10725-010-9488-7
25. Aerts R J, Gisi D, Carolis E De, Luca V De, Baumann T W (1994) Methyl jasmonate vapor increases the developmentally controlled synthesis of alkaloids in Catharanthus and Cinchona seedlings. Plant Cell 5:635-643
26. Vázquez-Flota F A, De Luca V (1998) Jasmonate modulates development- and light-regulated akaloid biosynthesis in Catharanthus roseus. Phytochemistry 49:395-402. doi.org/10.1016/S0031-9422(98)00176-9
27. Wang Q, Yuan F, Pan Q, U M, Wang G, Zhao J, Tang K (2010) Isolation and functional analysis of the Catharanthus roseus acetylindoline-4-O-acetytransferase gene promoter. Plant Cell Rep 29:185-192. doi.org/10.1007/s00299-009-0811-2
28. van der Fits L Van Der, Memelink J (2000) ORCA3, a Jasmonate-Responsive Transcriptional Regulator of Plant Primary and Secondary Metabolism. Science (80-) 289:295-298
29. Hernández-Dominguez E, Campos-Tamayo F, Vázquez-Flota F (2004) Vindoline synthesis in in vitro shoot cukures of Catharanthus roseus. Biotechnol Lett 26:671-674. doi.org/10.1023/B:BILE.0000023028.21985.07
30. Zhou P, Yang J, Zhu J, He S, Zhang W, Yu R, Z I J, Song L, Huang X (2015) Effects of β-cyclodextrin and methyl jasmonate on the production of vindoline, catharanthine, and ajmalicine in Catharanthus roseus cambial meristematic cel cultures. Appl Microbiol Biotechnol 99:7035-7045. doi.org/10.1007/s00253-015-651-9
31. Raina S K, Wankhede D P, Jaggi M, Singh P, Jalmi S K, Raghuram B, Sheikh A H, Sinha A K (2012) CrMPK3, a mitogen activated protein kinase from Catharanthus roseus and its possible role in stress induced biosynthesis of monoterpenoid indole alkaloids. BMC Plant Biol 12:134. doi.org/10.1186/1471-2229-12-134
32. Liscombe D K, Usera A R, O'Connor S E (2010) Homolog of tocopherol C methyltransferases catalyzes N methylation in anticancer alkaloid biosynthesis. Proc Natl Acad Sci USA. doi.org/10.1073/pnas.1009003107
33. Vázquez-Flota F, Carrilo-Pech M, Minero-Garcí a Y, de Lourdes Miranda-Ham M (2004) Alkaloid metabolism in wounded Catharanthus roseus seedlings. Plant Physiol Biochem 42:623-628. doi.org/doi.org/10.1016/j.plaphy.2004.06.010
34. Vázquez-Flota F, Hernández-Dominguez E, De Lourdes Miranda-Ham M, Monforte-González M (2009) A differential response to chemical elicitors in Catharanthus roseus In vitro cultures. Biotechnol Lett 31:591-595. doi.org/10.1007/s10529-008-98814
35. Pan Q, Chen Y, Wang Q, Yuan F, Xing S, Tian Y, Zhao J, Sun X, Kexuan T (2010) Effect of plant growth regulators on the biosynthesis of vinblastine, vindoline and catharanthine in Catharanthus roseus. Plant Growth Regul 60:133-141. doi.org/10.1007/s10725-009-9429-1
36. Pan Y jie, Lin Y chao, Yu B fan, Zu Y gang, Yu F, Tang Z H (2018) Transcriptomics comparison reveals the diversity of ethylene and methyl-jasmonate in roles of TIA metabolism in Catharanthus roseus. BMC Genomics 19:1-14. doi.org/10.1186/s12864-018-4879-3
37. El-Sayed M, Verpoorte R (2005) Methyljasmonate accelerates catabolism of monoterpenoid Indole alkaloids in Catharanthus roseus during leaf processing. Fitoterapia 76:83-90. doi.org/10.1016/j.fitote.2004.10.019
38. Gulrimand G, Courdavault V, Lanoue A, Mahroug S, Gulhur A, Blanc N, Giglioll-Gulvarc'h N, St-Pierre B, Burlat V (2010) Strictosidine activation in Apocynaceae: towards a “nuclear time bomb”? BMC Plant Biol 10:182. doi.org/10.1186/1471-2229-10-182
39. El-Sayed M, Verpoorte R (2004) Growth, metabolic profiling and enzymes activities of Catharanthus roseus seedings treated with plant growth regulators. Plant Growth Regul 44:53-58. doi.org/10.1007/s10725-004-2604-5
40. Bernonville T D De, Carqueijeiro I, Lanoue A, Lafontaine F, Bel P S, Liesecke F, Musset K, Oudin A, Glévarec G, Pichon O, Besseau S, Clastre M, St-pierre B, Flors V, Maury S, Huguet E, Connor S E O, Courdavault V (2017) Folivory elicits a strong defense reaction in Catharanthus roseus: metabolomic and transcriptomic analyses reveal distinct local and systemic responses. Nat Publ Gr 1-14. doi.org/10.1038/srep40453
41. Fernández-Mllmanda G L, Crocco C D, Reichel M, Mazza C A, Kölliner T G, Zhang T, Cargnel M D, Lichy M Z, Fiorucci A-S, Fankhauser C, Koo A J, Austin A T, Gershenzon J, Ballaré C L (2020) A light-dependent molecular link between competition cues and defence responses in plants. Nat Plants 6:223-230. doi.org/10.1038/s41477-020-0604-8
42. Chico J-M, Fernández-Barbero G, Chini A, Fernández-Calvo P, Díez-Diaz M, Solano R (2014) Repression of Jasmonate-Dependent Defenses by Shade Involves Differential Regulation of Protein Stability of MYC Transcription Factors and Their JAZ Repressors in Arabidopsis. Plant Cell 26:1967-1980. doi.org/10.1105/tpc.114.125047
43. Srivastava M, Srivastava A K, Roy D, Mansl M, Gough C, Bhagat P K, Zhang C, Sadanandom A (2022) The conjugation of SUMO to the transcription factor MYC2 functions in blue light-mediated seedling development in Arabidopsis. Plant Cell koac142. doi.org/10.1093/picell/koac142
44. Zhao P, Zhang X, Gong Y, Wang D, Xu D, Wang N, Sun Y, Gao L, Liu S-S, Deng X W, Kliebenstein D J, Zhou X, Fang R-X, Ye J (2021) Red-light is an environmental effector for mutualism between begomovirus and its vector whitefly. PLOS Pathog 17:e1008770
45. Zhang X, J I Y, Xue C, Ma H, X I Y, Huang P, Wang H, An F, U B, Wang Y, Guo H (2018) Integrated Regulation of Apical Hook Development by Transcriptional Coupling of EIN3/EIL1 and PIFs in Arabidopsis. Plant Cel 30:1971-1988. doi.org/10.1105/tpc.18.00018
46. Luo F, Zhang O, Xin H, Lu H, Yang H-Q, Doblin M S, Back A, Li L (2021) A Phytochrome B-PIF4-MYC2 Module Tunes Secondary Cel Wal Thickening in Response to Shaded Lighting. bioRxiv 2021.10.11.463962. doi.org/10.1101/2021.10.11.463962
47. Blanco-Tourdñán N, Serrano-Mislata A, Alabedl D (2020) Regulation of DELLA Proteins by Post-translational Modifications. Plant Cel Physiol 61:1891-1901. doi.org/10.1093/pcp/pcaa113
48. Daviére J-M, Achard P (2013) Glbberelin signaling in plants. Development 140:1147 LP-1151. doi.org/10.1242/dev.087650
49. Reid J B, Botwright N A, Smith J J, O'Nell D P, Kerckhoffs L H J (2002) Control of gibberellin levels and gene expression during de-etiolation in pea. Plant Physiol 128:734-741. doi.org/10.1104/pp.010607
50. Kamiya Y, Garcia-Martinez J L (1999) Regulation of gibberellin biosynthesis by light. Curr Opin Plant Biol 2:398-403. doi.org/doi.org/10.1016/S1369-5266(99)00012-8
51. Blanco-Tourdfin N, Legris M, Minguet E G, Costiglolo-Rojas C, Nohales M A, Iniesto E, Garcia-León M, Pacin M, Heucken N, Biomeier T, Locascio A, Černý M, Esteve-Bruna D, Diez-Diaz M, Brzobohatý B, Frerigmann H, Zurbriggen M D, Kay S A, Rubio V, Blázquez M A, Casal J J, Alabadi D (2020) COP1 destabilizes DELLA proteins in Arabidopsis. Proc Natl Acad Sci 117:13792 LP-13799. doi.org/10.1073/pnas.1907969117
52. Lee B-D, Yim Y, Cañibano E, Kim S-H, Garcia-León M, Rubio V, Fonseca S, Paek N-C (2022) CONSTITUTIVE PHOTOMORPHOGENIC 1 promotes seed germination by destabilizing RGA-LIKE 2 in Arabidopsis. Plant Physiol 189:1662-1676. doi.org/10.1093/plphys/kiac060
53. Li K, Yu R, Fan L-M, Wei N, Chen H, Deng X W (2016) DELLA-mediated PIF degradation contributes to coordination of light and gibberellin signaling in Arabidopsis. Nat Commun 7:11868. doi.org/10.1038/ncomms11868
54. de Lucas M, Davlbre J-M, Rodriguez-Falcón M, Pontin M, Iglesias-Pedraz J M, Lorrain S, Fankhauser C, Blázquez M A, Titarenko E, Prat S (2008) A molecular framework for light and gibberellin control of cel elongation. Nature 451:480-484. doi.org/10.1038/nature06520
55. Feng S, Martinez C, Gusmaroli G, Wang Y, Zhou J, Wang F, Chen L, Yu L, Iglesias-Pedraz J M, Kircher S, Schäfer E, Fu X, Fan L-M, Deng X W (2008) Coordinated regulation of Arabidopsis thaliana development by light and gibberellins. Nature 451:475-479 doi.org/10.1038/nature06448
56. Gallego-Bartolomé J, Minguet E G, Marin J A, Prat S, Blázquez M A, Alabadí D (2010) Transcriptional Diversification and Functional Conservation between DELLA Proteins in Arabidopsis. Mol Biol Evol 27:1247-1256. doi.org/10.1093/molbev/msq012
57. Achard P, Liao L, Jiang C, Desnos T, Bartlett J, Fu X, Harberd N P (2007) DELLAs Contribute to Plant Photomorphogenesis. Plant Physiol 143:1163-1172. doi.org/10.1104/pp.106.092254
58. Djakovio-Petrovic T, Wit M de, Voesenek L A C J, Plerik R (2007) DELLA protein function in growth responses to canopy signals. Plant J 51:117-126. doi.org/10.1111/j.1365-313X.2007.03122.x
59. Yang D-L, Yao J, Mel C-S, Tong X-H, Zeng L-J, Li Q, Xiao L-T, Sun T, Li J, Deng X-W, Lee C M, Thomashow M F, Yang Y, He Z, He S Y (2012) Plant hormone jasmonate prioritizes defense over growth by interfering with gibberellin signaling cascade. Proc Natl Aced Sci USA 109:E1192-E1200. doi.org/10.1073/pnas.1201616109
60. Heinrich M, Hettenhausen C, Lange T, Wünsche H, Fang J, Baldwin I T, Wu J (2013) High levels of jasmonic acid antagonize the biosynthesis of gibberellins and Inhibit the growth of Nicotiana attenuata stems. Plant J 73:591-606. doi.org/10.1111/tpj.12058
61. Sheerin D J, Menon C, zur Oven-Krockhaus S, Enderle B, Zhu L, Johnen P, Schielfenbaum F, Stierhof Y-D, Huq E, Hiltbrunner A (2015) Light-activated phytochrome A and B interact with members of the SPA family to promote photomorphogenesis in Arabidopsis by reorganizing the COP1/SPA complex. Plant Cell 27:189-201. doi.org/10.1105/tpc.114.134775
62. Lu X-D, Zhou C-M, Xu P-B, Luo Q, Lian H-L, Yang H-Q (2015) Red-light-dependent Interaction of phyB with SPA1 promotes COP1-SPA1 dissociation and photomorphogenic development in Arabidopsis. Mol Plant 8:487-478. doi.org/10.1016/j.molp.2014.11.025
63. Franke J, Kim J, Hamilton J P, Zhao D, Pham G M, Wiegert-Rininger K, Crisovan E, Newton L, Vaillancourt B, Tatsis E, Buell C R, O'Connor S E (2018) Gene discovery in Gelsemium highlights conserved gene clusters in monoterpene Indole akaloid biosynthesis-Dryad Digital Repository—Dryad
64. Itoh H, Ueguchl-Tanaka M, Sato Y, Ashikari M, Matsuoka M (2002) The Gibberellin Signaling Pathway Is Regulated by the Appearance and Disappearance of SLENDER RICE1 in Nuclei. Plant Cell 14:57-70. doi.org/10.1105/tpc.010319
65. Pysh L D, Wysocka-Diller J W, Camilleri C, Bouchez D, Benfey P N (1999) The GRAS gene family in Arabidopsis: sequence characterization and basic expression analysis of the SCARECROW-LIKE genes. Plant J 18:111-119. doi.org/10.1046/j.1365-313x.1999.00431.x
66. Gazara R K, Moharana K C, Bellieny-Rabelo D, Venancio T M (2018) Expansion and diversification of the gibberellin receptor GIBBERELLIN INSENSITIVE DWARF1 (GID1) family in land plants. Plant Mol Biol 97:435-449. doi.org/10.1007/s11103-018-0750-9
67. Zhang H (2008) Jasmonate-responsive transcriptional regulation in Catharanthus roseus (PhD Thesis). Leiden Univ
68. Fernandez-Pozo N, Rosli H G, Martin G B, Mueller L A (2015) The SGN VIGS Tool: User-Friendly Software to Design Virus-Induced Gene Silencing (VIGS) Constructs for Functional Genomics. Mol Plant 8:486-488, doi.org/doi.org/10.1016/j.molp.2014.11.024
69. Mortensen S, Bernal-Franco D, Cole L F, Sathitloetsakun S, Cram E J, Lee-Parsons C W T (2019) EASI Transformation: An Efficient Transient Expression Method for Analyzing Gene Function in Catharanthus roseus Seedlings. Front Plant Sci 10:1-17. doi.org/10.3389/fpls.2019.00755
70. Mortensen S (2019) Transcriptional Repression of Terpenoid Indole Akaloid Biosynthesis in Catharanthus roseus. Northeastern University
71. Gietz R D, Schiestl R H (2007) Quick and easy yeast transformation using the LiAc/S S carrier DNA/PEG method. Nat Protoc 2:35-37. doi.org/10.1038/nprot.2007.14
72. Cuéllar A P, Pauwels L, De Clercq R, Goossens A (2013) Yeast Two-Hybrid Analysis of Jasmonate Signaling Proteins. Humana Press, Totowa, NJ, pp 173-185
73. Liscombe D K, O'Connor S E (2011) A virus-induced gene silencing approach to understanding alkaloid metabolism in Catharanthus roseus. Phytochemistry 72:1969-77. doi.org/10.1016/j.phytochem.2011.07.001
74. Moreno J E, Tao Y, Chory J, Ballaré C L (2009) Ecological modulation of plant defense via phytochrome control of jasmonate sensitivity. Proc Natl Acad Sci 106:4935 LP-4940. doi.org/10.1073/pnas.0900701106
75. Goklany S, Rizvl N F, Loring R H, Cram E J, Lee-parsons CWT (2013) Jasmonate-Dependent Akaloid Biosynthesis in Catharanthus roseus Hairy Root Cultures Is Correlated with the Relative Expression of Orca and Zct Transcription Factors. 1367-1376. doi.org/10.1002/btpr.1801
76. Pollier J, Vanden Bossche R, Rischer H, Goossens A (2014) Selection and validation of reference genes for transcript normalization in gene expression studies in Catharanthus roseus. Plant Physiol Biochem 83:20-25. doi.org/10.1016/j.plaphy.2014.07.004
77. Livak K J, Schmittgen T D (2001) Analysis of relative gene expression data using real-time quantitative PCR and the 2-ΔΔCT method. Methods 25:402-408. doi.org/10.1006/meth.2001.1262
78. Svec D, Tichopad A, Novosadova V, Pfaffl M W, Kubista M (2015) How good is a PCR efficiency estimate: Recommendations for precise and robust qPCR efficiency assessments. Biomol Detect Quantf 3:9-16. doi.org/doi.org/10.1016/j.bdq.2015.01.005
79. Yoshida H, Hirano K, Sato T, Mitsuda N, Nomoto M, Maeo K, Koketsu E, Mitani R, Kawamura M, Ishiguro S, Tada Y, Ohme-Takagi M, Matsuoka M, Ueguchl-Tanaka M (2014) DELLA protein functions as a transcriptional activator through the DNA binding of the INDETERMINATE DOMAIN family proteins. Proc Natl Acad Sci USA. doi.org/10.1073/pnas.1321669111
80. Marin-de la Rosa N, Pfeiffer A, Hill K, Locascio A, Bhalerao R P, Miskolczi P, Grønlund A L, Wanchoo-Kohli A, Thomas S G, Bennett M J, Lohmann J U, Blázquez M A, Alabadi D (2015) Genome Wide Binding Site Analysis Reveals Transcriptional Coactivation of Cytokinin-Responsive Genes by DELLA Proteins. PLoS Genet 11:1-20. doi.org/10.1371/journal.pgen.1005337
81. Gallego-Bartolomé J, Minguet E G, Grau-Enguix F, Abbas M, Locascio A, Thomas S G, Alabadi D, Blázquez M A (2012) Molecular mechanism for the Interaction between gibberellin and brassinosteroid signaling pathways in Arabidopsis. Proc Natl Acad Sci USA 109:13448-13451. doi.org/10.1073/pnas.1119992109
82. Hou X, Lee L Y C, Xia K, Yan Y, Yu H (2010) DELLAs Modulate Jasmonate Signaling via Competitive Binding to JAZs. Dev Cell 19:884-894. doi.org/10.1016/j.devcel.2010.10.024
83. Jiang Y, Chen J, Zheng X, Tan B, Ye X, Wang W, Zhang L, Li J, U Z, Cheng J, Feng J (2022) Multiple Indeterminate domain (IDD)-DELLA1 complexes participate in gibberelin feedback regulation in peach. Plant Mol Biol. doi.org/10.1007/s11103-022-01263-y
84. Murase K, Hirano Y, Sun T, Hakoshima T (2008) Gibberellin-induced DELLA recognition by the gibberellin receptor GID1. Nature 456:459-483, doi.org/10.1038/nature07519
85. Alyssa D, Hou-Sung J, Tai-ping S (2001) The DELLA motif is essential for gibberellin-induced degradation of RGA. Proc Natl Acad Sci 98:14162-14167. doi.org/10.1073/pnas.251534098
86. Muangprom A, Thomas S G, Sun T-P, Osborn T C (2005) A novel dwarfing mutation in a green revolution gene from Brassica rapa. Plant Physiol 137:931-938. doi.org/10.1104/pp.104.057648
87. Hirano K, Asano K, Tsuji H, Kawamura M, Mor H, Kitano H, Ueguchl-Tanaka M, Matsuoka M (2010) Characterization of the molecular mechanism underlying gibberellin perception complex formation in rice. Plant Cell 22:2680-2696. doi.org/10.1105/tpc.110.075549
88. Peng J, Carol P, Richards D E, King K E, Cowing R J, Murphy G P, Harberd N P (1997) The Arabidopsis GAI gene defines a signaling pathway that negatively regulates gibberellin responses. Genes Dev 11:3194-3205. doi.org/10.1101/grad.11.23.3194
89. Cassani E, Bertoini E, Cerino Badone F, Landoni M, Gavina D, Srizzotti A, Pilu R (2009) Characterization of the first dominant dwarf maize mutant carrying a single amino acid insertion i the VHYNP domain of the dwarf8 gene. Mol Breed 24:375. doi.org/10.1007/s11032-009-9298-3
90. Winkler R G, Freeling M (1994) Physiological genetics of the dominant gibberellin-nonresponsive maize dwarfs, Dwarf5 and Dwarf9. Planta 193:341-348. doi.org/10.1007/BF00201811
91. Boss P K, Thomas M R (2002) Association of dwarfism and floral Induction with a grape ‘green revolution’ mutation. Nature 416:847-850. doi.org/10.1038/416847a
92. Peng J, Richards D E, Hartley N M, Murphy G P, Devos K M, Flintham J E, Beales J, Fish L, Worland A J, Pelica F, Sudhakar D, Christou P, Snape J W, Gale M D, Harberd N P (1999) ‘Green revolution’ genes encode mutant gibberellin response modulators. Nature 400:256-261. doi.org/10.1038/22307
93. Harberd N P, Freeing M (1989) Genetics of dominant gibberellin-Insensitive dwarfism in maize. Genetics 121:827-838. doi.org/10.1093/genetics/121.4.827
94. Hirano K, Kouketu E, Katoh H, Aya K, Ueguchl-Tanaka M, Matsuoka M (2012) The suppressive function of the rice dela protein SLR1 is dependent on its transcriptional activation activity. Plant J 71:443-453. doi.org/10.1111/j.1365-313X.2012.05000.x
95. Phokas A, Coates J C (2021) Evolution of DELLA function and signaling in land plants. Evol Dev 23:137-154. doi.org/doi.org/10.1111/ede.12385
96. Weston D E, Elliott R C, Lester D R, Rameau C, Reid J B, Murfet I C, Ross J J (2008) The Pea DELLA Proteins L A and CRY Are Important Regulators of Gibberellin Synthesis and Root Growth. Plant Physiol 147:199-205. doi.org/10.1104/pp.108.115808
97. Franke J, Kim J, Hamilkon J P, Zhao D, Pham G M, Wiegert-Rininger K, Crisovan E, Newton L, Vaillancourt B, Tatsis E, Buell C R, O'Connor S E (2019) Gene discovery in Gelsemium highlights conserved gene clusters in monoterpene Indole alkaloid biosynthesis. ChemBioChem 20:83-87. doi.org/10.1002/cbic.201800592
98. Zhang Y, Turner J G (2008) Wound-Induced Endogenous Jasmonates Stunt Plant Growth by Inhibiting Miosis. PLoS One 3:e3699
99. Liu G, Yin K, Zhang O, Gao C, Qiu J-L (2019) Modulating chromatin accessibly by transactivation and targeting proximal dsgRNAs enhances Cas9 editing efficiency in vivo. Genome Biol 20:145. doi.org/10.1186/s13059-019-1762-8
100. Casal J J (2012) Shade avoidance. Arab B 10:e0157-e0157. doi.org/10.1199/tab.0157
101. Küpers J J, Snoek B L, Oskam L, Pantazopoulou C K, Matton S E A, Reinen E, Liao C-Y, Eggermont E D C, Weekamp H, Kohlen W, Weijers D, Pierik R (2022) Local light signaling at the leaf tip drives remote differential petiole growth through auxin-gibberellin dynamics. bioRxiv 2022.02.25.481815. doi.org/10.1101/2022.02.25.481815
102. Srivastava N K, Srivastava A K (2007) Influence of gibberellic acid on 14CO2 metabolism, growth, and production of alkaloids in Catharanthus roseus. Photosynthetica 45:156-160. doi.org/10.1007/s11099-007-0026-0
103. Zhong M, Zeng B, Tang D, Yang J, Qu L, Yan J, Wang X, Li X, Liu X, Zhao X (2021) The blue light receptor CRY1 Interacts with GID1 and DELLA proteins to repress GA signaling during photomorphogenesis in Arabidopsis. Mol Plant 14:1328-1342. doi.org/doi.org/10.1016/j.molp.2021.05.011
104. Xu P, Chen H, Li T, Xu F, Mao Z, Cao X, Miao L, Du S, Hua J, Zhao J, Guo T, Kou S, Wang W, Yang H-Q (2021) Blue light-dependent Interactions of CRY1 with GID1 and DELLA proteins regulate gibberellin signaling and photomorphogenesis in Arabidopsis. Plant Cell 33:2375-2394. doi.org/10.1093/picell/koab124
105. Alabadi D, Gil J, Blázquez M A, Garcia-Martinez J L (2004) Gibberellins repress photomorphogenesis in darkness. Plant Physiol 134:1050-1057. doi.org/10.1104/pp.103.035451
106. Cheminant S, Wild M, Bouvier F, Pelletier S, Renou J-P, Erhardt M, Hayes S, Terry M J, Genschik P, Achard P (2011) DELLAs regulate chlorophyll and carotenoid biosynthesis to prevent photooxidative damage during seedling deetiolation in Arabidopsis. Plant Cel 23:1849-1860. doi.org/10.1105/tpc.111.085233
107. Dill A, Thomas S G, Hu J, Steber C M, Sun T-P (2004) The Arabidopsis F-box protein SLEEPY1 targets gibberellin signaling repressors for gibberellin-induced degradation. Plant Cel 16:1392-1405. doi.org/10.1105/tpc.020958
108. Kamran M, Wennan S, Ahmad I, Xiangping M, Wenwen C, Xudong Z, Siwel M, Khan A, Qingfang H, Tiening L (2018) Application of paclobutrazol affect maize grain yield by regulating root morphological and physiological characteristics under a semi-arid region. Sci Rep 8:4818. doi.org/10.1038/s41598-018-23166-z
109. Hedden P (2003) The genes of the Green Revolution. Trends Genet 19:5-9. doi.org/10.1016/s0168-9525(02)00009-4
110. Mall M, Singh P, Kumar R, Shanker K, Gupta A K, Khare P, Shasany A K, Khatoon S, Sundaresan V, Baskaran K, Yadav S, Shukla A K (2021) Phenotypic, genetic and expression profiling of a vindoline-rich genotype of Catharanthus roseus. South African J Bot 139:50-57 doi.org/doi.org/10.1016/j.sajb.2021.02.004
111. Chung I-M, Kim E-H, Li M, Peebles CAM, Jung W-S, Song H-K, Ahn J-K, San K-Y (2011) Screening 64 cultivars Catharanthus roseus for the production of vindoline, catharanthine, and serpentine. Biotechnol Prog 27:937-943. doi.org/10.1002/btpr.557
112. Helden R, Jacobs D, Snoeijer W, Hallard D, Verpoorte R (2005) The Catharanthus Alkaloids: Pharmacognosy and Biotechnology. Curr Med Chem 11:607-628. doi.org/10.2174/0929867043455848
113. Kulkarni R N, Baskaran K, Chandrashekara R S, Kumar S (1999) Inheritance of morphological traits of periwinkle mutants with modified contents and yields of leaf and root alkaloids. Plant Breed 118:71-74. doi.org/doi.org/10.1046/j.1439-0523.1999.118001071.x
114. Verma A K, Singh R R (2010) Induced Dwarf Mutant in Catharanthus roseus with Enhanced Antibacterial Activity. Indian J Pharm Sci 72:655-657. doi.org/10.4103/0250-474X.78541
115. Shaw S E (2015) The Effect of Light, Jasmonate, and Tissue Organization on the Expression of Vindoline Pathway Genes in Catharanthus Roseus. Northeastern University
116. Liscombe D K, O'Connor S E (2011) A virus-induced gene silencing approach to understanding alkaloid metabolism in Catharanthus roseus. Phytochemistry 72:1969-1977. doi.org/10.1016/j.phytochem.2011.07.001
117. Traverse†,*, K. K. F., Mortensen†,*, S., Trautmann‡, J. G., Danison‡, H., Rizvi, N. F., Lee-Parsons, C. W. T. (2022) “Generation of stable Catharanthus roseus hairy root lines with Agrobacterium rhizogenes.” In: Fett-Neto, A. G. (ads) Plant Secondary Metabolism Engineering. Methods in Molecular Biology, vol 2469. Humana, New York, NY. doi.org/10.1007/978-1-0716-2185-1_11
118. Rizvi†, N. F., Cornejo‡, M., Stein‡, K., Weaver†, J., Cram, E. J., Lee-Parsons, C. W. T. (2015) “An efficient transformation method for estrogen-inducible transgene expression in Catharanthus roseus hairy roots,” Plant Cell, Tissue and Organ Culture (PCTOC), 120(2): 475-487. doi.org/10.1007/s11240-014-0614-1
119. Rizvi†, N. F., Weaver†, J., Cram, E. J., Lee-Parsons, C. W. T. (2016) “Silencing the transcriptional repressor, ZCT1, illustrates the tight regulation of terpenoid indole alkaloid biosynthesis”, PLoS ONE, 11(7):e0159712. doi.org/10.1371/journal.pone.0159712
120. Grützner, R., Martin, P., Horn, C., Mortensent, S., Cram, E. J., Lee-Parsons, C. W. T., Stuttmann, J., Marillonnet, S. (2021) “High-efficiency genome editing in plants mediated by a Cas9 gene containing multiple introns,” Plant Communications 2(2), 100135. doi.org/10.1016/j.xplc.2020.100135
121. Rafael Zarate, Robert Verpoorte (2007) Strategies for the genetic modification of the medicinal plant Catharanthus roseus (L.) G. Don. Phytochem Rev (2007) 6:475-491
122. Verma, P, Khan, S. A., Mathur, A. K. (2022) De Novo Shoot Bud Induction from the Catharanthus roseus Leaf Explants and Agrobacterium tumefaciens-Mediated Technique to Raise Transgenic Plants. In: Courdavauk, V., Besseau, S. (eds) Catharanthus roseus. Methods in Molecular Biology, vol 2505. Humana, New York, NY.
123. Bomzan, D. P., Shilpashree, H. B., Nagegowda, D. A. (2022) Agrobacterium-Mediated in Planta Transformation in Periwinkle. In: Courdavauk, V., Besseau, S. (eds) Catharanthus roseus. Methods in Molecular Biology, vol 2505. Humana, New York, NY.

1. A Catharanthus roseus (C. roseus) plant comprising one or more DELLA transcription factors having enhanced activity compared to a naturally occurring C. roseus plant, wherein the plant is capable of enhanced production of vindoline compared to said naturally occurring C. roseus plant; wherein the plant is a transgenic plant or a plant obtained by selective breeding.

2. (canceled)

3. (canceled)

4. The plant of claim 1, wherein the plant has a higher level of CrDELLA1 and/or CrDELLA2 proteins than said naturally occurring C. roseus plant.

5. (canceled)

6. (canceled)

7. The plant of claim 1, wherein one or more DELLA transcription factors activate a biosynthetic pathway leading to vindoline synthesis.

8. The plant of claim 7, wherein the one or more DELLA transcription factors activate a biosynthetic pathway from geraniol and tryptophan to tabersonine (i.e., terpenoid indole alkaloid (TIA) pathway) as well as a biosynthetic pathway from tabersonine to vindoline (vindoline pathway).

9. The plant of claim 7, wherein said one or more DELLA transcription factors bind and inhibit a jasmonate zim domain protein (JAZ) and/or a phytochrome interacting factor protein (PIF).

10. The plant of claim 1, wherein the plant comprises a CrDELLA1 and/or CrDELLA2 gene harboring a gain of function mutation.

11. The plant of claim 10, wherein the gain of function mutation is selected from mutations that inhibit gibberellic acid insensitive dwarf 1a (GID1a) and/or gibberellic acid insensitive dwarf 1b (GID1b) binding to DELLA, mutations that inhibit degradation of DELLA in the presence of gibberellic acid, and mutations that disrupt DELLA-COP1 binding.

12. The plant of claim 11, wherein the gain of function mutation inhibits GID1a and/or GID1b from binding to DELLA, and wherein the mutation comprises an N-terminal deletion of up to 112 amino acids of CrDELLA1 and/or CrDELLA2.

13. The plant of claim 1, wherein CrGID1a and/or CrGID1b protein intracellular levels are reduced in the plant.

14. The plant of claim 13, wherein the CrGID1a and/or CrGID1b genes are knocked down or knocked out in the plant.

15. (canceled)

16. The plant of claim 1, wherein the plant is also capable of enhanced production of vinblastine and/or vincristine upon processing leaves of the plant to promote reaction of vindoline and catharanthine, compared to said naturally occurring C. roseus plant.

17. A method of preparing a C. roseus plant capable of enhanced vindoline production, the method comprising the steps of:
(a) introducing a gain of function mutation in a CrDELLA1 gene and/or a CrDELLA2 gene into a C. roseus plant; and/or
(b) reducing the ability of a CrGID1a protein and/or a CrGID1b protein to cause degradation of CrDELLA1 protein and/or CrDELLA2 protein in said C. roseus plant; whereby the C. roseus plant becomes capable of enhanced vindoline production.

18. (canceled)

19. The method of claim 17, wherein the gain of function mutation comprises deleting up to 112 amino acids at the N-terminus of CrDELLA1 and/or CrDELLA2.

20. The method of claim 17, wherein point mutations in a CrDELLA1, CrDELLA2, CrGID1a, and/or CrGID1b gene are introduced by radiation or chemical agent mutagenesis or by selective breeding combined with screening for increased levels of CrDELLA1 protein and/or CrDELLA2 protein.

21. (canceled)

22. A method of producing vinblastine and/or vincristine, the method comprising the steps of:
(a) providing the plant of claim 1; and
(b) growing the plant under conditions suitable for the production of precursors of vinblastine and/or vincristine in the plant.

23. The method of claim 22, further comprising contacting the transgenic plant with a compound that reduces the level of gibberellic acid in the plant.

24. (canceled)

25. (canceled)

26. The method of claim 2226, further comprising contacting the plant with a plant defense hormone.

27. (canceled)

28. (canceled)

29. The method of claim 22, further comprising the steps of:
(c) subjecting leaves of the plant to a treatment that enhances alkaloid biosynthesis in leaves of the plant; and
(d) waiting for a period of time, during which vindoline and catharanthine accumulate in said leaves.

30. The method of claim 29, further comprising the steps of:
(e) harvesting said leaves;
(f) homogenizing the harvested leaves in a buffer solution, whereby said vindoline and catharanthine are released from cells of the harvested leaves and one or more enzymes involved in biosynthesis of vincristine and/or vinblastine are also released from cells of the harvested leaves; and
(g) incubating the homogenized leaves, whereby vincristine and/or vinblastine are produced from reaction of said vindoline and catharanthine.

31. A cell obtained from the plant of claim 1, or a C. roseus cell bearing identical genetic modifications compared to said plant.


SEQUENCE LISTING (82 sequences)

SEQ ID NO: 1 - protein, 587 aa, Arabidopsis thaliana
MKRDHHQFQGRLSNHGTSSSSSSISKDKMMMVKKEEDGGGNMDDELLAVLGYKVRSSEMAEVALKLEQLETMMSNVQEDGLSHLATDTVHYNPSELYSWLDNMLSELNPPPLPASSNGLDPVLPSPEICGFPASDYDLKVIPGNAIYQFPAIDSSSSSNNQNKRLKSCSSPDSMVTSTSTGTQIGGVIGTTVTTTTTTTTAAGESTRSVILVDSQENGVRLVHALMACAEAIQQNNLTLAEALVKQIGCLAVSQAGAMRKVATYFAEALARRIYRLSPPQNQIDHCLSDTLQMHFYETCPYLKFAHFTANQAILEAFEGKKRVHVIDFSMNQGLQWPALMQALALREGGPPTFRLTGIGPPAPDNSDHLHEVGCKLAQLAEAIHVEFEYRGFVANSLADLDASMLELRPSDTEAVAVNSVFELHKLLGRPGGIEKVLGVVKQIKPVIFTVVEQESNHNGPVFLDRFTESLHYYSTLFDSLEGVPNSQDKVMSEVYLGKQICNLVACEGPDRVERHETLSQWGNRFGSSGLAPAHLGSNAFKQASMLLSVFNSGQGYRVEESNGCLMLGWHTRPLITTSAWKLSTAAY

SEQ ID NO: 2 - protein, 533 aa, Arabidopsis thaliana
MKRDHHHHHHQDKKTMMMNEEDDGNGMDELLAVLGYKVRSSEMADVAQKLEQLEVMMSNVQEDDLSQLATETVHYNPAELYTWLDSMLTDLNPPSSNAEYDLKAIPGDAILNQFAIDSASSSNQGGGGDTYTTNKRLKCSNGVVETTTATAESTRHVVLVDSQENGVRLVHALLACAEAVQKENLTVAEALVKQIGFLAVSQIGAMRKVATYFAEALARRIYRLSPSQSPIDHSLSDTLQMHFYETCPYLKFAHFTANQAILEAFQGKKRVHVIDFSMSQGLQWPALMQALALRPGGPPVFRLTGIGPPAPDNFDYLHEVGCKLAHLAEAIHVEFEYRGFVANTLADLDASMLELRPSEIESVAVNSVFELHKLLGRPGAIDKVLGVVNQIKPEIFTVVEQESNHNSPIFLDRFTESLHYYSTLFDSLEGVPSGQDKVMSEVYLGKQICNVVACDGPDRVERHETLSQWRNRFGSAGFAAAHIGSNAFKQASMLLALFNGGEGYRVEESDGCLMLGWHTRPLIATSAWKLSTN

SEQ ID NO: 3 - protein, 511 aa, Arabidopsis thaliana
MKREHNHRESSAGEGGSSSMTTVIKEEAAGVDELLVVLGYKVRSSDMADVAHKLEQLEMVLGDGISNLSDETVHYNPSDLSGWVESMLSDLDPTRIQEKPDSEYDLRAIPGSAVYPRDEHVTRRSKRTRIESELSSTRSVVVLDSQETGVRLVHALLACAEAVQQNNLKLADALVKHVGLLASSQAGAMRKVATYFAEGLARRIYRIYPRDDVALSSFSDTLQIHFYESCPYLKFAHFTANQAILEVFATAEKVHVIDLGLNHGLQWPALIQALALRPNGPPDFRLTGIGYSLTDIQEVGWKLGQLASTIGVNFEFKSIALNNLSDLKPEMLDIRPGLESVAVNSVFELHRLLAHPGSIDKFLSTIKSIRPDIMTVVEQEANHNGTVFLDRFTESLHYYSSLFDSLEGPPSQDRVMSELFLGRQILNLVACEGEDRVERHETLNQWRNRFGLGGFKPVSIGSNAYKQASMLLALYAGADGYNVEENEGCLLLGWQTRPLIATSAWRINRVE

SEQ ID NO: 4 - protein, 547 aa, Arabidopsis thaliana
MKRGYGETWDPPPKPLPASRSGEGPSMADKKKADDDNNNSNMDDELLAVLGYKVRSSEMAEVAQKLEQLEMVLSNDDVGSTVLNDSVHYNPSDLSNWVESMLSELNNPASSDLDTTRSCVDRSEYDLRAIPGLSAFPKEEEVFDEEASSKRIRLGSWCESSDESTRSVVLVDSQETGVRLVHALVACAEAIHQENLNLADALVKRVGTLAGSQAGAMGKVATYFAQALARRIYRDYTAETDVCAAVNPSFEEVLEMHFYESCPYLKFAHFTANQAILEAVTTARRVHVIDLGLNQGMQWPALMQALALRPGGPPSFRLTGIGPPQTENSDSLQQLGWKLAQFAQNMGVEFEFKGLAAESLSDLEPEMFETRPESETLVVNSVFELHRLLARSGSIEKLLNTVKAIKPSIVTVVEQEANHNGIVFLDRFNEALHYYSSLFDSLEDSYSLPSQDRVMSEVYLGRQILNVVAAEGSDRVERHETAAQWRIRMKSAGFDPIHLGSSAFKQASMLLSLYATGDGYRVEENDGCLMIGWQTRPLITTSAWKLA

SEQ ID NO: 5 - protein, 523 aa, Arabidopsis thaliana
MKRSHQETSVEEEAPSMVEKLENGCGGGGDDNMDEFLAVLGYKVRSSDMADVAQKLEQLEMVLSNDIASSSNAFNDTVHYNPSDLSGWAQSMLSDLNYYPDLDPNRICDLRPITDDDECCSSNSNSNKRIRLGPWCDSVTSESTRSVVLIEETGVRLVQALVACAEAVQLENLSLADALVKRVGLLAASQAGAMGKVATYFAEALARRIYRIHPSAAAIDPSFEEILQMNFYDSCPYLKFAHFTANQAILEAVTTSRVVHVIDLGLNQGMQWPALMQALALRPGGPPSFRLTGVGNPSNREGIQELGWKLAQLAQAIGVEFKFNGLTTERLSDLEPDMFETRTESETLVVNSVFELHPVLSQPGSIEKLLATVKAVKPGLVTVVEQEANHNGDVFLDRFNEALHYYSSLFDSLEDGVVIPSQDRVMSEVYLGRQILNLVATEGSDRIERHETLAQWRKRMGSAGFDPVNLGSDAFKQASLLLALSGGGDGYRVEENDGSLMLAWQTKPLIAASAWKLAAELRR

SEQ ID NO: 6 - protein, 653 aa, Arabidopsis thaliana
MAESGDFNGGQPPPHSPLRTTSSGSSSSNNRGPPPPPPPPLVMVRKRLASEMSSNPDYNNSSRPPRRVSHLLDSNYNTVTPQQPPSLTAAATVSSQPNPPLSVCGFSGLPVFPSDRGGRNVMMSVQPMDQDSSSSSASPTVWVDAIIRDLIHSSTSVSIPQLIQNVRDIIFPCNPNLGALLEYRLRSLMLLDPSSSSDPSPQTFEPLYQISNNPSPPQQQQQHQQQQQQHKPPPPPIQQQERENSSTDAPPQPETVTATVPAVQTNTAEALRERKEEIKRQKQDEEGLHLLTLLLQCAEAVSADNLEEANKLLLEISQLSTPYGTSAQRVAAYFSEAMSARLLNSCLGIYAALPSRWMPQTHSLKMVSAFQVFNGISPLVKFSHFTANQAIQEAFEKEDSVHIIDLDIMQGLQWPGLFHILASRPGGPPHVRLTGLGTSMEALQATGKRLSDFADKLGLPFEFCPLAEKVGNLDTERLNVRKREAVAVHWLQHSLYDVTGSDAHTLWLLQRLAPKVVTVVEQDLSHAGSFLGRFVEAIHYYSALFDSLGASYGEESEERHVVEQQLLSKEIRNVLAVGGPSRSGEVKFESWREKMQQCGFKGISLAGNAATQATLLLGMFPSDGYTLVDDNGTLKLGWKDLSLLTASAWTPRS

SEQ ID NO: 7 - protein, 625 aa, Oryza sativa
MKREYQEAGGSSGGGSSADMGSCKDKVMAGAAGEEEDVDELLAALGYKVRSSDMADVAQKLEQLEMAMGMGGVSAPGAADDGFVSHLATDTVHYNPSDLSSWVESMLSELNAPLPPIPPAPPAARHASTSSTVTGGGGSGFFELPAAADSSSSTYALRPISLPVVATADPSAADSARDTKRMRTGGGSTSSSSSSSSSLGGGASRGSVVEAAPPATQGAAAANAPAVPVVVVDTQEAGIRLVHALLACAEAVQQENFAAAEALVKQIPTLAASQGGAMRKVAAYFGEALARRVYRFRPADSTLLDAAFADLLHAHFYESCPYLKFAHFTANQAILEAFAGCHRVHVVDFGIKQGMQWPALLQALALRPGGPPSFRLTGVGPPQPDETDALQQVGWKLAQFAHTIRVDFQYRGLVAATLADLEPFMLQPEGEADANEEPEVIAVNSVFELHRLLAQPGALEKVLGTVHAVRPRIVTVVEQEANHNSGSFLDRFTESLHYYSTMFDSLEGGSSGQAELSPPAAGGGGGTDQVMSEVYLGRQICNVVACEGAERTERHETLGQWRNRLGRAGFEPVHLGSNAYKQASTLLALFAGGDGYRVEEKEGCLTLGWHTRPLIATSAWRVAAA

SEQ ID NO: 8 - protein, 630 aa, Zea mays
MKREYQDAGGSGGDMGSSKDKMMAAAAGAGEQEEEDVDELLAALGYKVRSSDMADVAQKLEQLEMAMGMGGVGGAGATADDGFVSHLATDTVHYNPSDLSSWVESMLSELNAPPAPLPPATPAPRLASTSSTVTSGAAAGAGYFDLPPAVDSSSSTYALKPIPSPVAAPSADPSTDSAREPKRMRTGGGSTSSSSSSSSSMDGGRTRSSVVEAAPPATQASAAANGPAVPVVVVDTQEAGIRLVHALLACAEAVQQENFSAAEALVKQIPMLASSQGGAMRKVAAYFGEALARRVYRFRPPPDSSLLDAAFADLLHAHFYESCPYLKFAHFTANQAILEAFAGCRRVHVVDFGIKQGMQWPALLQALALRPGGPPSFRLTGVGPPQPDETDALQQVGWKLAQFAHTIRVDFQYRGLVAATLADLEPFMLQPEGDDTDDEPEVIAVNSVFELHRLLAQPGALEKVLGTVRAVRPRIVTVVEQEANHNSGTFLDRFTESLHYYSTMFDSLEGAGAGSGQSTDASPAAAGGTDQVMSEVYLGRQICNVVACEGAERTERHETLGQWRSRLGGSGFAPVHLGSNAYKQASTLLALFAGGDGYRVEEKDGCLTLGWHTRPLIATSAWRVAAAAAP

SEQ ID NO: 9 - protein, 625 aa, Zea mays
MKREYQNAGGNDGYRGSSKDKSMAAAAGAGEQEEEVDELLAALGYKVRSSDMADVAQKLEQLEMAMGMGGACPTADDGFVSHLATDTVHYNPSDLSSWVESMLSELNAPPPPLPPATPAPRLASTSSTVTSGAAAGAGYFDLPPAVDSSSSTYALKPIPSPVAAASADPSPDSAREPKRMRTGGGSTSSSSSSSSSMDGGRTRSSVVEAAPPATQAANGPAVPVVVVDTQEAGIRLVHALLACAEAVQQENFSAADALVKQIPVLASSQGGAMRKVAAYFGEALARRVYRLRPAPDGSLLDAAFADLLHAHFYESCPYLKFAHFTANQAILEAFAGCRRVHVVDFGIKQGMQWPALLQALALRPGGPPSFRLTGVGPPQPDETDALQQVGWKLAQFAHTIRVDFQYRGLVAATLADLEPFMLRPEGGGDTDDEPEVIAVNSVCELHRLLAQPGTLDKVLGTVRAVRPRIVTVVEQEANHNSGTFLDRFTESLHYYSTMFDSLEGAGSGSGSGSGSGQPTDASPPAGTDQVMSEVYLGRQICNIVACEGAERTERHETLVQWRGRLGGSGFEPVHLGSNAYKQASTLLALFAGGDGYRVEEKDGCLTLGWHTRPLIATSAWRVAAP

SEQ ID NO: 10 - protein, 588 aa, Solanum lycopersicum
MKRDRDRDREREKRAFSNGAVSSGKSKIWEEDEEEKPDAGMDELLAVLGYKVKSSDMADVAQKLEQLEMAMGTTMEDGITHLSTDTVHKNPSDMAGWVQSMLSSISTNFDMCNQENDVLVSGCGSSSSIIDFSQNHRTSTISDDDLRAIPGGAVFNSDSNKRHRSTTSSFSTTSSSMVTDSSATRPVVLVDSQETGVRLVHTLMACAEAVQQENLTLADQLVRHIGILAVSQSGAMRKVATYFAEALARRIYKIYPQDSMESSYTDVLQMHFYETCPYLKFAHFTANQAILEAFTGCNKVHVIDFSLKQGMQWPALMQALALRPGGPPAFRLTGIGPPQPDNTDALQQVGWKLAQLAETIGVEFEFRGFVANSLADLDATILDIRPSETEAVAINSVFELHRLLSRPGAIEKVLNSIKQINPKIVTLVEQEANHNAGVFIDRFNEALHYYSTMFDSLESSGSSSSASPTGILPQPPVNNQDLVMSEVYLGRQICNVVACEGSDRVERHETLNQWRVRMNSSGFDPVHLGSNAFKQASMLLALFAGGDGYRVEENDGCLMLGWHTRPLIATSAWKLLPDSGTGAGEVEL

SEQ ID NO: 11 - protein, 623 aa, Triticum aestivum
MKREYQDAGGSGGGGGGMGSSEDKMMVSAAAGEGEEVDELLAALGYKVRASDMADVAQKLEQLEMAMGMGGVGAGAAPDDSFATHLATDTVHYNPTDLSSWVESMLSELNAPPPPLPPAPQLNASTSSTVTGSGGYFDLPPSVDSSSSIYALRPIPSPAGATAPADLSADSVRDPKRMRTGGSSTSSSSSSSSSLGGGARSSVVEAAPPVAAAANATPALPVVVVDTQEAGIRLVHALLACAEAVQQENLSAAEALVKQIPLLAASQGGAMRKVAAYFGEALARRVFRFRPQPDSSLLDAAFADLLHAHFYESCPYLKFAHFTANQAILEAFAGCRRVHVVDFGIKQGMQWPALLQALALRPGGPPSFRLTGVGPPQPDETDALQQVGWKLAQFAHTIRVDFQYRGLVAATLADLEPFMLQPEGEEDPNEEPEVIAVNSVFEMHRLLAQPGALEKVLGTVRAVRPRIVTVVEQEANHNSGTFLDRFTESLHYYSTMFDSLEGGSSGGGPSEVSSGAAAAPAAAGTDQVMSEVYLGRQICNVVACEGAERTERHETLGQWRNRLGNAGFETVHLGSNAYKQASTLLALFAGGDGYKVEEKEGCLTLGWHTRPLIATSAWRLAGP

SEQ ID NO: 12 - protein, 618 aa, Hordeum vulgare
MKREYQDGGGSGGGGDEMGSSRDKMMVSSSEAGEGEEVDELLAALGYKVRASDMADVAQKLEQLEMAMGMGGPAPDDGFATHLATDTVHYNPTDLSSWVESMLSELNAPPPPLPPAPPQLNASTSSTVTGGGGYFDLPPSVDSSSSTYALRPIISPPVAPADLSADSVRDPKRMRTGGSSTSSSSSSSSSLGGGAARSSVVEAAPPVAAAAAAPALPVVVVDTQEAGIRLVHALLACAEAVQQENLSAAEALVKQIPLLAASQGGAMRKVAAYFGEALARRVFRFRPQPDSSLLDAAFADLLHAHFYESCPYLKFAHFTANQAILEAFAGCRRVHVVDFGIKQGMQWPALLQALALRPGGPPSFRLTGVGPPQPDETDALQQVGWKLAQFAHTIRVDFQYRGLVAATLADLEPFMLQPEGEEDPNEEPEVIAVNSVFEMHRLLAQPGALEKVLGTVRAVRPRIVTVVEQEANHNSGSFLDRFTESLHYYSTMFDSLEGGSSGGPSEVSSGGAAPAAAAGTDQVMSEVYLGRQICNVVACEGTERTERHETLGQWRNRLGNAGFETVHLGSNAYKQASTLLALFAGGDGYKVEEKEGCLTLGWHTRPLIATSAWRLAAP

SEQ ID NO: 13 - protein, 592 aa, Pisum sativum
MKRDRQETSGGGVSSNNNNSDNIISNNFVSVRGECSSMQNNNNNGKQSLWSEEKENSGGMDELLAALGYKVRSSDMLDVAQKLEQLEMVMGSAQEEGINHLASDTVHYDPTDLYSWVQTMLTELNPSTDSQINDPLDSSSSIFNDNSQYDLSVIPGMAAYPPQSHNQNSESEGSNSNKRLKTWGSETESEDIFLPALSPPAETTRPVVLVDSQETGVRLIHTMMACADAIQRDDIKIADRLVKNIGILASSQTGAMGKVASYFAQALYRRICRVSPDETLDSSLSDALHMHFYESSPYLKFAHFTANQAILEAFAGAGSVHVIDFGLKQGMQWPALMQALALRPGGPPTFRLTGIGPPQTGNTDALQQVGWKLAQLAQTIGVQFEFRGFVCNSLADLDPNMLEIRPGEAVAVNSVFELHTMLARPGSIDKVLNTVKKINPKIVTIVEQEANHNGPVFMDRFTEALHYYSSLFDSLEGSSNSNPAGSGSSSQDLLMSELYLGRQICNVVAYEGVDRVERHETLSQWRSRMGSAGFDPVHLGSNAFKQASTLLALFAGGDGYRVEENNGCLMLGWHTRSLIATSAWKLPLSESK

SEQ ID NO: 14 - protein, 532 aa, Pisum sativum
MKREHNQEHEDASMTGKSEICWEDDGGMDELLAVVGYKVKSSDMAEVAQKLEQLEQAMGNFQDQDEATIAQHLSNDTVHYNPADISNWLQTMLSNFDSQPNPSVSSSSDNDLNAIPGKAIYANTDSQTEESLPSRKRVKRVGSSSSTESTRPVVMVVETQEKGIILVHTLMACAEAVEQNNRPVAEALVKQIGNLAVSQEGAMRKVATYFAIGLARRIYDVFPQHSVSDSLQIHFYETCPYLKFAHFTANQAILEAFQGKSRVHVIDFSINQGMQWPALMQALALRPGGPPAFRLTGIGPPASDNSDHLQQVGWRLAQFAQTIHVQFEYRGFVANSLADLDASMLELRSPETESVAVNSVFELHKLNARPGALEKVFSVIRQIRPEIVTVVEQEANHNGPAFLDRFTESLHYYSTLFDSLESSLVEPQDKAMSEVYLGKQICNVVACEGTDRVERHETLNQWRNRFGSAGFSPVHLGSNAFKQASMLLALFAGGDGYKVEENDGCLMLGWHTRPLIATSAWKLAANSMVVSH

SEQ ID NO: 15 - protein, 590 aa, Vitis vinifera
MKREYHHPHHPTCSTSPTGKGKMWDADPQQDAGMDELLAVLGYNVKASDMAEVAQKLEQLEEVIVNAQEDGLSHLASETVHYNPSDLSNWLGSMLSEFNPTPNCALDNPFLPPISPLDYTNCSTQPKQEPSIFDSPSLDYDLKAIPGKALYSHIEQPPQQPPAPPLYQRDNKRLKPTTSATANSVSSVIGGWGVPTESARPVVLVDSQETGIRLVHTLMACAEAVQQENLKLAEALVKQIGFLAVSQAGAMRKVATYFAEGLARRIYRLYPDKPLDSSFSDILQMHFYETCPYLKFAHFTANQAILEAFEGKKRVHVIDFSMKQGMQWPALMQALALRPGGPPSFRLTGIGPPSTDNTDHLHEVGWKLAQLAETIHVEFEYRGFVANSLADLDASMLELRDGESVAVNSVFELHSLLARPGGIERVLSAVKDMKPDIVTIVEQEANHNGPVFLDRFTESLHYYSTLFDSLEGCGVSPVNTQDKLMSEVYLGQQICNVVACEGPERVERHETLAQWRARLGSAGFDPVNLGSNAFKQASMLLALFAGGDGYRVEENNGCLMLGWHTRPLIATSAWQLANKPALPSSTPASN

SEQ ID NO: 16 - protein, 573 aa, Brassica rapa
MKRDLHQFQGPNHGTSIAGSSTSSPAVFGKDKMMMVKEEEDDELLGVLGYKVRSSEMAEVALKLEQLETMMGNAQEDGLAHLATDTVHYNPAELYSWLDNMLTELNPPAATTGSNALNPEINNNNNNNSFFTGGDLKAIPGNAVCRRSNQFAFAVDSSSNKRLKPSSSPDSMVTSPSPAGVIGTTVTTVTESTRPLILVDSQDNGVRLVHALMACAEAVQSSNLTLAEALVKQIGFLAVSQAGAMRKVATYFAEALARRIYRLSPPQTQIDHSLSDTLQMHFYETCPYLKFAHFTANQAILEAFEGKKRVHVIDFSMNQGLQWPALMQALALREGGPPSFRLTGIGPPAADNSDHLHEVGCKLAQLAEAIHVEFEYRGFVANSLADLDASMLELRPSETEAVAVNSVFELHKLLGRTGGIEKVFGVVKQIKPVIFTVVEQESNHNGPVFLDRFTESLHYYSTLFDSLEGAPSSQDKVMSEVYLGKQICNLVACEGPDRVERHETLSQWSNRFGSSGFAPAHLGSNAFKQASTLLALFNGGEGYRVEENNGCLMLSWHTRPLITTSAWKLSAVH

SEQ ID NO: 17 - protein, 578 aa, Brassica rapa
MKRDLHQFQGPPDTRFPNHGTANTGSSSKDKMMMVKEEEDGGNMDELLAVLGYKVRSSEMAEVALKLEQLETMMGNVQEDGLSNLATDTVHYNPSELYSWLDNMLTEFNPPPPEINNSFLAGAGGSDYDLKAIPGNAIYARSDQFAIDSSSSSNQAGDNSQSTKRLKSCSSPDSLVTGTTVTTTTTESTRSVGLAAESTRSMVLVDSQENGVRLVHALMACAEAIQNNDLSIAEALVKQIGFLAVSQAGAMRKVATYFAEALARRIYRLSPPQTQIDHSLSDTLQMHFYETCPYLKFAHFTANQAILEAFEGKKRVHVIDFSMNQGLQWPALMQALALREGGPPVFRLTGIGPPAADNSDHLHEVGCKLAQLAEAIHVEFEYRGFVANSLADLDASMLELRPSEIEAVAVNSVFELHKLLGRTGGIEKVLGVVKQIKPVIFTVVEQESSHNGPVFLDRFTESLHYYSTLFDSLEGVPSSQDKVMSEVYLGKQICNLVACEGPDRVERHETLSQWANRFGSSGFAPAHLGSNAFKQASMLLALFNGGEGYRVEENNGCLMLGWHTRPLITTSAWKLSAA

SEQ ID NO: 18 - protein, 620 aa, Catharanthus roseus
MKRDHTKTQAAAQESFPGGGGSSTASGSCYTMGSSTSGMAKMWKDTVKQDAGDDELLALLGYKVKASDMPEVAQKIEQLEEFMGNVQQDGLSHLASDTVHYNPSDLYSWLESMISEINPDGSSLPDSSTITSLDNSSSTNTSQNNRGLSNGVLVFDDSSFDSDLTAIPGNAVYPQIQRSPPSTRLQPPSSSAQPPVVQPPNKRMKPNSQMGVFGIPNESVLPISRPVDSTRPVVLVDSQENGIRLVHTLMACAEAIQQENLKVAEALVKQIRFLAVSQAGAMRKVATYFAEALARRIYRLYPTNPHDSASTDLLQMHFYETCPYLKFAHFTANQAILEAFANKNRVHVIDFSIKQGLQWPALLQALALRPGGPPSFRLTGIGPPSHDNTDHLQEVGWKLAQLAETINVEFEYRGFVANSLADLDASMFDIREGETVAVNSIFELHQLLARQGAIDKVLSVVKELKPEIMTVVEQEANHNGPVFMDRFNESLHYYSTLFDSLEGCGNGGAGGGEGGASCGSPTDQDKVMSELYLGRQICNVVACEGVDRVERHETLTQWKTRFNSAGFAAVHLGSNAFKQASMLLALFAGGDGYRVEENNGCLMLGWHTRPLIATSAWKLG

SEQ ID NO: 19 - protein, 623 aa, Catharanthus roseus
MKRDHTKTQPAAAQESFSGGGGSSTASGSCYTMGSSNSGMAKMWKDTVKQDAGDDELLAVLGYKVKSSDMAEVAQKIEQLEEFMGNVQQDGLSHLASDTVHYNPSDLSSWLESMISEINPDGSSLPDSSTITSLDNSSSTNTSQNNINRGLSNGVLVFDDSSFDSDLTAIPGNAVYPQIHPSPPPTRLQPPSSSAQPPVVQPPNKRMKPNSQMGVFGIPNESVLPIARPMDSTRPVVLVDSQENGIRLVHALMACAEAIQQENLKVAEALVKQIGFLAVSQAGAMRKVATYFAEALARRIYRLYPTNPHDSAFTDLLQMHFYETCPYLKFAHFTANQAILEAFANNNRVHVIDFSIKQGLQWPALLQALALRPGGPPSFRLTGIGPPSHDNTDHLQEVGWKLAQLAETINVEFEYRGFVANSLADLDASMFDIREGETVAVNSIFELHQLLARQGAIDKVLSVVKELKPEIMTVVEQEANHNGPVFMDRFNESLHYYSTLFDSLEGCGNGGAGGGEGGASGGSPTDQDKVMSEVYLGRQICNVVACEGVDRVERHETLTQWKTRFNSAGFASVHLGSNAFKQASMLLALFAGGDGYRVEENNGCLMLGWHTRPLIATSAWKLG

SEQ ID NO: 20 - protein, 422 aa, Catharanthus roseus
MECHFLFLFAAKTPGEINHFLARVYSFGLSKVTNLRSSENFIIQNQTHFSQHIANEVVHYNPSNIGLWANCFSSELSQPSLPVFPDPNFGSTSTIGLKELPLTALQLNFSSLIFQRMKREQEDSSIQLIHVMITCAGTIQRGNLSLACTLVEKLQGLLACMDSTSTIGKASSYFIDALNLRMFTASQGINSXTFLPENEILYQNYYEATPYLKLANFTTNKAILEAFXGHDFVHVIHFSLMQGLQWPTLIQALALHSGGPPLLRITSVGPPSTNNNDSLGEIGFKLAVFARSINVNLAFRGVVASRLDDIRPWLFQTGQGEAIAVNSILELHKLIGLNSIHRSPIELVLNRQEVDHNLAEFLDRFTEVLFYYWTKFDLLEAFSAQLEKGLAELYMKREICNILSREGPAQTERHEPLANWRD

SEQ ID NO: 21 - protein, 347 aa, Catharanthus roseus
MDSTSVIGKASSYFIDALNLRMFTASQGINSXTFLPENEILYQNYYEATPYLKLANFTTNKAILEAFXGHDFVHVIHFSLMQGLQWPSLIHALALRPEGPPLLRITGILSDRDSVGEIINANLAFRGVVASKLDDINPWLLQTSQGEAIVVNSILELHXLIGLNSIHGSPIELVLNWVRNLNPNVITIVEQEVDHNQAEFLDRFILLFDHVLLVGGLFDSTEESSTAASNGSSSSCFATRTLDEINHFLARLCSSGLSKVTNNLRGSENFIIQNPPHFSQHLANDIVHYNPSNLGLWINSFQSKLSQSSLPVFPDPNFGSTSTIGLKELPLMAPQQNFSRLIFQRLK

SEQ ID NO: 22 - protein, 354 aa, Oryza sativa
MAGSDEVNRNECKTVVPLHTWVLISNFKLSYNILRRADGTFERDLGEYLDRRVPANARPLEGVSSFDHIIDQSVGLEVRIYRAAAEGDAEEGAAAVTRPILEFLTDAPAAEPFPVIIFFHGGSFVHSSASSTIYDSLCRRFVKLSKGVVVSVNYRRAPEHRYPCAYDDGWTALKWVMSQPFMRSGGDAQARVFLSGDSSGGNIAHHVAVRAADEGVKVCGNILLNAMFGGTERTESERRLDGKYFVTLQDRDWYWKAYLPEDADRDHPACNPFGPNGRRLGGLPFAKSLIIVSGLDLTCDRQLAYADALREDGHHVKVVQCENATVGFYLLPNTVHYHEVMEEISDFLNANLYY

SEQ ID NO: 23 - protein, 345 aa, Arabidopsis thaliana
MAASDEVNLIESRTVVPLNTWVLISNFKVAYNILRRPDGTFNRHLAEYLDRKVTANANPVDGVFSFDVLIDRRINLLSRVYRPAYADQEQPPSILDLEKPVDGDIVPVILFFHGGSFAHSSANSAIYDTLCRRLVGLCKCVVVSVNYRRAPENPYPCAYDDGWIALNWVNSRSWLKSKKDSKVHIFLAGDSSGGNIAHNVALRAGESGIDVLGNILLNPMFGGNERTESEKSLDGKYFVTVRDRDWYWKAFLPEGEDREHPACNPFSPRGKSLEGVSFPKSLVVVAGLDLIRDWQLAYAEGLKKAGQEVKLMHLEKATVGFYLLPNNNHFHNVMDEISAFVNAEC

SEQ ID NO: 24 - protein, 358 aa, Arabidopsis thaliana
MAGGNEVNLNECKRIVPLNTWVLISNFKLAYKVLRRPDGSFNRDLAEFLDRKVPANSFPLDGVFSFDHVDSTTNLLTRIYQPASLLHQTRHGTLELTKPLSTTEIVPVLIFFHGGSFTHSSANSAIYDTFCRRLVTICGVVVVSVDYRRSPEHRYPCAYDDGWNALNWVKSRVWLQSGKDSNVYVYLAGDSSGGNIAHNVAVRATNEGVKVLGNILLHPMFGGQERTQSEKTLDGKYFVTIQDRDWYWRAYLPEGEDRDHPACNPFGPRGQSLKGVNFPKSLVVVAGLDLVQDWQLAYVDGLKKTGLEVNLLYLKQATIGFYFLPNNDHFHCLMEELNKFVHSIEDSQSKSSPVLLTP

SEQ ID NO: 25 - protein, 358 aa, Arabidopsis thaliana; identical to SEQ ID NO: 24

SEQ ID NO: 26 - protein, 335 aa, Arabidopsis thaliana
MATDSQPNQKLTLPLKTRIALTVISTMTDNAQRPDGTINRRFLRLFDFRAPPNPKPVNIVSTSDFVVDQSRDLWFRLYTPHVSGDKIPVVVFFHGGGFAFLSPNAYPYDNVCRRFARKLPAYVISVNYRLAPEHRYPAQYDDGFDALKYIEENHGSILPANADLSRCFFAGDSAGGNIAHNVAIRICREPRSSFTAVKLIGLISIQPFFGGEERTEAEKQLVGAPLVSPDRTDWCWKAMGLNRDHEAVNVGGPNAVDISGLDYPETMVVVAGFDPLKDWQRSYYEWLKLCGKKATLIEYPNMFHAFYIFPELPEAGQLIMRIKDFVDERVASLSA

SEQ ID NO: 27 - protein, 346 aa, Catharanthus roseus
MATGNEVNLNDSRRVVPLNTWVLISNFKLAYNLLRRPDGTFNRHLAEFLDRKVSANAIPVEGVYSFDVIIDREIGLLSRVYRPVQNDKAPSSFAELEQPVTADVVPVILFFHGGSFAHSSANSAIYDTLCRRLVRICKAVVISVNYRLAPENRYPCAYDDCWAAVKWANSRKWLQSKKDSKVHIFLCGDSSGGNIVHNVALRATESDIDILGNILLNPLFGGEKRTESELRLDGKYFVRVQDRDWYWRAYLPEDADRDHPACNPFGPNGINLKGVKFPKSLVVVAGLDLVQDWQRAYVDGLRDAGQEVNLISLEEATVGFYFLPNNDYFYIVMDAISDFVCAKSQS

SEQ ID NO: 28 - protein, 345 aa, Catharanthus roseus
MAGSNEINASESKRVVPLNTWILISNFKLAYNMLRRPDGTFNRELAEFLDRKVPANTIPVDGVYSFDVVDRTTSLLCRVYRPAPENEDQWGKIELEKPLSTTEIIPVILFFHGGSFAHSSANSAIYDTLCRRLVNICKAVVVSVDYRRSPEYRYPSAYEDGWAALKWVQSRVWLQSGRDSRVHVYLAGDSSGGNIIHHVAVRAADEGGVEVLGNILLQPLFGGETRTESEKRLDGKYFVKLQDRDWYWRAFLPEGEDRDHPACNVFGPRGRNLGGMKFPKSLVVVPGLDLLQDWQLSYVEGLKKYGQDVKLLFLKEATIGFYFLPNNDYFYTLMEEIKNFIHPYS

SEQ ID NO: 29 - protein, 327 aa, Catharanthus roseus
MDANKGTSLPSPALPLLTRFVIKLINGVTGAASRRNGTVNRRLLSILNYTSPANPNPIKGVSSIDVTVDPDRDLWFRIFVPTIDDWENLPVIVFFHGGGFAFLSPDVKSYDAVCRRFARKLPAVVVSVNYRLSPEHRYPAQIEDGYDVLKFLDDEKNKGILPANADVARCFLAGDSAGANLAHHVAVVGMVSIQPFFGGEERTEAEIKLSGIDPLVCTNRTDWLWNAYLPAGEGMNRDHEAINVSGPRAVDISGKEFPTTLVVVGGFDSLKDWQKRYCEYLHKSGKQVQLLEYPTMVHAFYVFPELKESAELISEIKDFVHNICSKL

SEQ ID NO: 30 - protein, 71 aa, Arabidopsis thaliana
DDELLAVLGYKVRSSEMAEVALKLEQLETMMSNVQEDGLSHLATDTVHYNPSELYSWLDNMLSELNPPPLP

SEQ ID NO: 31 - DNA, 49 bp, synthetic construct
  source 1..49: mol_type = other DNA
ggggacaagtttgtacaaaaaagcaggctggatgaagagggaccatacg

SEQ ID NO: 32 - DNA, 48 bp, synthetic construct
  source 1..48: mol_type = other DNA
ggggaccactttgtacaagaaagctgggtagtttaaccgagtttccag

SEQ ID NO: 33 - DNA, 50 bp, synthetic construct
  source 1..50: mol_type = other DNA
ggggacaagtttgtacaaaaaagcaggctctatgtatccttgctttcctg

SEQ ID NO: 34 - DNA, 48 bp, synthetic construct
  source 1..48: mol_type = other DNA
ggggaccactttgtacaagaaagctgggtacttcagcctagactttgc

SEQ ID NO: 35 - DNA, 49 bp, synthetic construct
  source 1..49: mol_type = other DNA
ggggacaagtttgtacaaaaaagcaggctcgatggataaatctggccag

SEQ ID NO: 36 - DNA, 49 bp, synthetic construct
  source 1..49: mol_type = other DNA
ggggaccactttgtacaagaaagctgggtgggttttaaaaaggaaagcc

SEQ ID NO: 37 - DNA, 34 bp, synthetic construct
  source 1..34: mol_type = other DNA
aagaagacaaaatgaagagggaccatacgaaaac

SEQ ID NO: 38 - DNA, 34 bp, synthetic construct
  source 1..34: mol_type = other DNA
aagaagacaaaagcttaaccgagtttccaggccg

SEQ ID NO: 39 - DNA, 29 bp, synthetic construct
  source 1..29: mol_type = other DNA
aagaagacaaaatgaagagggaccatacg

SEQ ID NO: 40 - DNA, 31 bp, synthetic construct
  source 1..31: mol_type = other DNA
aagaagacaacgaaccaccgagtttccaggc

SEQ ID NO: 41 - DNA, 34 bp, synthetic construct; identical to SEQ ID NO: 37
  source 1..34: mol_type = other DNA

SEQ ID NO: 42 - DNA, 31 bp, synthetic construct; identical to SEQ ID NO: 40
  source 1..31: mol_type = other DNA

SEQ ID NO: 43 - DNA, 33 bp, synthetic construct
  source 1..33: mol_type = other DNA
tttggtctcatatgacccttttcgattcacttg

SEQ ID NO: 44 - DNA, 34 bp, synthetic construct
  source 1..34: mol_type = other DNA
tttggtctcaatttcactctatagccatctccac

SEQ ID NO: 45 - DNA, 30 bp, synthetic construct
  source 1..30: mol_type = other DNA
tttggtctctaaattcgctcactttacagc

SEQ ID NO: 46 - DNA, 32 bp, synthetic construct
  source 1..32: mol_type = other DNA
tttggtctcacacctgcatcaagatcagcaag

SEQ ID NO: 47 - DNA, 38 bp, synthetic construct
  source 1..38: mol_type = other DNA
tttggtctcatatgctatttgtatgatatcaagattcg

SEQ ID NO: 48 - DNA, 34 bp, synthetic construct
  source 1..34: mol_type = other DNA
tttggtctcaggcaatgccaacttgaaatttgag

SEQ ID NO: 49 - DNA, 29 bp, synthetic construct
  source 1..29: mol_type = other DNA
tttggtctcttgccgtagattagttaaca

SEQ ID NO: 50 - DNA, 34 bp, synthetic construct
  source 1..34: mol_type = other DNA
tttggtctcacacctgattctgttcttgtttctc

SEQ ID NO: 51 - DNA, 19 bp, synthetic construct
  source 1..19: mol_type = other DNA
gccttcacgaatatcaaac

SEQ ID NO: 52 - DNA, 18 bp, synthetic construct
  source 1..18: mol_type = other DNA
taagcttggactcccatg

SEQ ID NO: 53 - DNA, 26 bp, synthetic construct
  source 1..26: mol_type = other DNA
tgtatcgataattgtaaatgtaattg

SEQ ID NO: 54 - DNA, 20 bp, synthetic construct
  source 1..20: mol_type = other DNA
atgatttctgaaatcaaccc

SEQ ID NO: 55 - DNA, 27 bp, synthetic construct
  source 1..27: mol_type = other DNA
aagaagacaaaatgggcgtttttggga

SEQ ID NO: 56 - DNA, 31 bp, synthetic construct; identical to SEQ ID NO: 40
  source 1..31: mol_type = other DNA

SEQ ID NO: 57 - DNA, 31 bp, synthetic construct
  source 1..31: mol_type = other DNA
atgggcgtttttgggattccaaatgagtctg

SEQ ID NO: 58 - DNA, 27 bp, synthetic construct
  source 1..27: mol_type = other DNA
ccagcctgcttttttgtacaaagttgg

SEQ ID NO: 59 - DNA, 21 bp, synthetic construct
  source 1..21: mol_type = other DNA
gctgaggctatccaacaagaa

SEQ ID NO: 60 - DNA, 21 bp, synthetic construct
  source 1..21: mol_type = other DNA
gccaatgcttcagcgaaatag

SEQ ID NO: 61 - DNA, 22 bp, synthetic construct
  source 1..22: mol_type = other DNA
ggtatggcaaagatgtggaaag

SEQ ID NO: 62 - DNA, 20 bp, synthetic construct
  source 1..20: mol_type = other DNA
cagccatgtccgaggattta

SEQ ID NO: 63 - DNA, 18 bp, synthetic construct
  source 1..18: mol_type = other DNA
ttgctgggctgctgtaaa

SEQ ID NO: 64 - DNA, 23 bp, synthetic construct
  source 1..23: mol_type = other DNA
ccaccagaactatcaccacataa

SEQ ID NO: 65 - DNA, 21 bp, synthetic construct
  source 1..21: mol_type = other DNA
ctaacttcaagctggcctaca

SEQ ID NO: 66 - DNA, 23 bp, synthetic construct
  source 1..23: mol_type = other DNA
gaattgtgttagcagggactttc

SEQ ID NO: 67 - DNA, 21 bp, synthetic construct
  source 1..21: mol_type = other DNA
gatcaactcacagtggcagtc

SEQ ID NO: 68 - DNA, 21 bp, synthetic construct
  source 1..21: mol_type = other DNA
gacttgagacttgtgattggc

SEQ ID NO: 69 - DNA, 23 bp, synthetic construct
  source 1..23: mol_type = other DNA
gtgtgaagatactcaaaagctgc

SEQ ID NO: 70 - DNA, 26 bp, synthetic construct
  source 1..26: mol_type = other DNA
caaaatttacaagcattgccatatcc

SEQ ID NO: 71 - DNA, 22 bp, synthetic construct
  source 1..22: mol_type = other DNA
gtcatagacgagcacagagaaa

SEQ ID NO: 72 - DNA, 22 bp, synthetic construct
  source 1..22: mol_type = other DNA
caccaccctcttcaatcctaag

SEQ ID NO: 73 - DNA, 22 bp, synthetic construct
  source 1..22: mol_type = other DNA
cttgagccactctttgctttac

SEQ ID NO: 74 - DNA, 20 bp, synthetic construct
  source 1..20: mol_type = other DNA
atgagggacattgcggatac

SEQ ID NO: 75 - DNA, 24 bp, synthetic construct
  source 1..24: mol_type = other DNA
tgacaaagtaaccggagcatggga

SEQ ID NO: 76 - DNA, 24 bp, synthetic construct
  source 1..24: mol_type = other DNA
atccgaatgacggcatcttggcta

SEQ ID NO: 77 - DNA, 20 bp, synthetic construct
  source 1..20: mol_type = other DNA
tggcctcagtagcaattcag

SEQ ID NO: 78 - DNA, 23 bp, synthetic construct
  source 1..23: mol_type = other DNA
tccatatttctcactcgcttctc

SEQ ID NO: 79 - DNA, 23 bp, synthetic construct
  source 1..23: mol_type = other DNA
gaggttttgactgcttttctcag

SEQ ID NO: 80 - DNA, 20 bp, synthetic construct
  source 1..20: mol_type = other DNA
tggaaatggcaaagattggc

SEQ ID NO: 81 - DNA, 20 bp, synthetic construct
  source 1..20: mol_type = other DNA
tgctgtggaggaggaagaag

SEQ ID NO: 82 - DNA, 21 bp, synthetic construct
  source 1..21: mol_type = other DNA
actggcggaactactactacc